(19) United States
(12) Patent Application Publication
(10) Pub. No.: US 2026/0194516 A1
(43) Pub. Date: Jul. 9, 2026

(54) BIOMARKERS PREDICTIVE OF IMMUNOMODULATORY MESENCHYMAL STROMAL CELLS

(71) Applicant: DRK-Blutspendedienst Baden-Württemberg-Hessen gGmbH, Frankfurt am Main (DE)

(72) Inventors: Hanna BÖHM, Weiterstadt (DE); Richard SCHÄFER, Buggingen (DE); Erhard SEIFRIED, Frankfurt am Main (DE); Georg SIEGEL, Norderstedt (DE); Gabriele SPOHN, Suizbach am Main (DE)

(21) Appl. No.: 19/131,701

(22) PCT Filed: Nov. 23, 2023
(86) PCT No.: PCT/EP2023/082871

§ 371 Date: May 21, 2025

(30) Foreign Application Priority Data
Nov. 23, 2022 (EP) 22209228.0

(51) Int. Cl. G01N 33/50

(52) U.S. Cl. CPC G01N 33/5073; G01N 33/5023; G01N 2500/10

(57) ABSTRACT

The invention is based on a novel technology to identify inflammatory responsive elements (IREs) in a mesenchymal stromal cell (MSC) culture that allowed for the development of biomarkers, in particular CD55, CD146, CD271 and MSCA-1, or combinations thereof. The biomarkers of the invention are indicative for an immunomodulatory activity of an MSC. Consequently, the present invention pertains a first aspect, to a method for determining a presence or absence of an immunomodulatory activity of a mesenchymal stromal cell (MSC). Furthermore, in a second aspect, a method for the manufacture of an immunomodulatory preparation derived from an MSC with immunomodulatory activity is disclosed. In a third and fourth aspect, the invention pertains to an immunomodulatory preparation derived from an MSC with immunomodulatory activity and its medical use. In further aspects, the invention also pertains to methods for identifying an immunomodulatory population and an immunomodulatory subpopulation of MSCs with a known presence and/or expression of a biomarker respectively, and to an agent for determining a presence or absence of an immunomodulatory activity of an MSC.

### FIELD OF THE INVENTION

The invention is based on a novel technology to identify inflammatory responsive elements (IREs) in a mesenchymal stromal cell (MSC) culture that allowed for the development of biomarkers, in particular CD55, CD146, CD271 and MSCA-1, or combinations thereof. The biomarkers of the invention are indicative for an immunomodulatory activity of an MSC. Consequently, the present invention pertains a first aspect, to a method for determining a presence or absence of an immunomodulatory activity of a mesenchymal stromal cell (MSC). Furthermore, in a second aspect, a method for the manufacture of an immunomodulatory preparation derived from an MSC with immunomodulatory activity is disclosed. In a third and fourth aspect, the invention pertains to an immunomodulatory preparation derived from an MSC with immunomodulatory activity and its medical use. In further aspects, the invention also pertains to methods for identifying an immunomodulatory population and an immunomodulatory subpopulation of MSCs with a known presence and/or expression of a biomarker respectively, and to an agent for determining a presence or absence of an immunomodulatory activity of an MSC.

### DESCRIPTION

Mesenchymal stem/stromal cells (MSCs) are stromal cells with multilineage differentiation potential that are known for self-renewing differentiation capacity and immunomodulatory properties. They are distinguished from other cell types by expression of cell surface markers including CD73, CD90, and CD105, and lack of expression of CD45, CD34, CD14, CD19, CD11b, or low Human Leukocyte Antigen-DR isotype (HLA-DR) expression (steady state conditions). Furthermore, MSCs are reported to play a role in tissue repair in addition to their potential anti-tumorigenic, anti-fibrotic, anti-apoptotic, anti-inflammatory, pro-angiogenic, neuroprotective, anti-bacterial and chemo-attractive effects. This unique set of characteristics make MSCs attractive for their therapeutic potential in the fields of regenerative medicine, inflammatory disorder, and cancer therapy.

While human bone marrow-derived MSCs (hBM-MSCs) feature a promising potential for clinical immunotherapy, substantial interdonor variabilities and unclear functional heterogeneity of MSC subpopulations hamper their sustainable clinical implementation. A specific challenge is the absence of potency markers that can reliably predict the MSCs' clinical efficacy.

US2016120910A1 and US2017136065A1 disclose methods for treating rheumatoid arthritis or sepsis patients comprising the use of mesenchymal stromal cells. US2012027730A1 discloses the use of mesenchymal stem cells (MSCs) for treating systemic inflammatory response syndrome (SIRS) in a subject. These applications characterize various MSC surface markers. However, there is no indication that the markers therein may be used for the identification of immunomodulatory MSC populations, or as surrogate for the immunomodulatory potential of an MSC preparation.

To trigger production of immunomodulating factors such as the indoleamine 2,3-dioxygenase (IDO)-1, MSCs may need to be exposed to inflammatory environments. Thus, the inventors hypothesized that MSC phenotypes that are particularly responsive to inflammation (IREs) could act as key elements in the MSC preparations immunomodulation potential, and, thus, utilizing the response of MSCs to inflammation could reveal MSC surface markers as potential surrogate for the MSCs' immunomodulation potency.

The inventors performed gene expression screenings, which revealed a substantial impact of inflammatory environment on the up- or down regulation of IRE mRNAs of hBM-MSCs. Based on these findings, and validating the RNA screening on protein level, the inventors were able to identify surface proteins such as CD55, CD146, CD271 and MSCA-1 that can act as biomarkers for MSC preparations with high immunomodulatory potential. In case of CD55, they further identified an IDO-1 mediated mode of action.

CD55 is also known as complement decay accelerating factor. Studies on the CD55 protein include are Soland et al. (doi: 10.1371/journal.pone.0060461), wherein CD46, CD55 and CD59 proteins are described as allowing MSCs to be able to inhibit activation of the complement system to a certain extent. Gavin et al. (doi: 10.3389/fimmu.2019.02249) focuses on the protection of MSCs against complement by the expression of complement inhibitory receptors CD46, CD55 and CD59.

However, none of the prior art documents discloses predictive biomarkers that are useful for identifying MSC populations with immunomodulatory activity. Hence, the object of the present invention is providing one or more biomarkers that allow such identification of MSC or MSC populations having immunomodulatory activity and/or potential.

The present invention solves this problem in a generic aspect by providing any one or a combination of biomarkers selected from CD55, CD146, MSCA-1 and CD271. In a first aspect a method for determining the presence or absence of an immunomodulatory activity of a mesenchymal stromal cell. Generally, the invention in the generic aspect and in any of the below captioned specific aspects is predicated upon the development of the biomarkers of the invention, in particular CD55, CD146, MSCA-1, and/or CD271. The biomarkers of the invention are particularly advantageous since biomarker detection and predictive value is independent of whether the analysed MSC or MSC population is activated (pre-treated with inflammatory stimuli), and hence can be applied to analyse MSCs under steady state conditions (e.g. CD55 and CD146).

### BRIEF DESCRIPTION OF THE INVENTION

Generally, and by way of brief description, the main aspects of the present invention can be described as follows:

In a first aspect, the invention pertains to a method for determining a presence or absence of an immunomodulatory activity of a mesenchymal stromal cell (MSC), wherein the method comprises a step of: detecting a presence and/or an expression of at least one biomarker selected from the group of CD55, CD271, MSCA-1 and CD146 associated with the MSC, preferably wherein the presence and/or expression of the biomarker is detected: within the MSC, attached to the MSC, or released from the MSC; wherein the presence and/or expression of the biomarker associated with the MSC is indicative for an immunomodulatory activity of the MSC.

In a second aspect, the invention pertains to a method for the manufacture of an immunomodulatory preparation derived from an MSC with immunomodulatory activity.

In a third aspect, the invention pertains to an immunomodulatory preparation derived from an MSC with immunomodulatory activity, preferably as detected in accordance with the invention, wherein the preparation comprises:
(i) the MSC with immunomodulatory activity,
(ii) a cell, vesicle, a micelle and/or a compound according to any aspect of the invention, and/or
(iii) a medium from a culture of the MSC with immunomodulatory activity.

In a fourth aspect, the invention pertains to an immunomodulatory MSC preparation according to any aspect of the invention, for use in the treatment of a condition or disease.

In a fifth aspect, the invention pertains to a method for identifying an immunomodulatory population of MSCs with a known presence and/or expression of a biomarker, comprising:
(i) co-culturing an MSC population, and allogeneic activated PBMNCs, or selected immune cell subsets, in separate or joint compartments with shared media; and
(ii) detecting a presence and/or expression of a biomarker of the co-cultured MSC population, preferably by transcriptome analysis and/or proteome analysis and comparing said presence and/or expression of the biomarker of the co-cultured MSC population with the presence and/or expression of the biomarker mono-cultured MSCs of the MSC population; and
(iii) detecting a presence and/or expression of an immunomodulating factor in the co-culture, preferably by transcriptome analysis and/or proteome analysis and comparing said presence and/or expression of immunomodulating factor in the co-culture with the presence and/or expression of the immunomodulating factor in a culture of mono-cultured MSCs of the MSC population; and/or
(iv) detecting the proliferation inhibition of the co-cultured allogeneic activated PBMNCs, or selected immune cell subsets, and comparing said proliferation inhibition with the proliferation of allogeneic activated PBMNCs, or selected immune cell subsets, that are cultured in a separate culture that does not share media with the MSC population;
   wherein an increased presence of the immunomodulating factor, an increased expression of the immunomodulating factor and/or an increased proliferation inhibition of the allogeneic activated PBMNCs, or selected immune cell subsets, indicates an immunomodulatory MSC population; and
(v) phenotyping the MSC population regarding the presence and/or expression of said biomarker,
thus, resulting in the identification of immunomodulatory population of MSCs with a known presence and/or expression of a biomarker.

In a sixth aspect, the invention pertains to a method for identifying an immunomodulatory subpopulation of MSCs with a known presence and/or expression of a biomarker, wherein the method is the method according to any one of the previous claims, wherein the immunomodulatory population of MSCs is an immunomodulatory subpopulation of MSCs and wherein the method comprises an additional step of functional validation of an immunomodulatory activity of a subpopulation of MSCs depleted of the biomarker.

In a seventh aspect, the invention pertains to an agent for determining a presence or absence of an immunomodulatory activity of an MSC, wherein the agent specifically binds to a biomarker selected from the group of CD55, CD271, MSCA-1 and CD146.

### DETAILED DESCRIPTION OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, the invention pertains to a method for determining a presence or absence of an immunomodulatory activity of a mesenchymal stromal cell (MSC), wherein the method comprises a step of: detection of a presence and/or an expression of at least one biomarker selected from the group of CD55, CD271, MSCA-1 and CD146 associated with the MSC, wherein the presence and/or expression of the biomarker associated with the MSC is indicative for an immunomodulatory activity of the MSC.

In the context of the invention, the at least one biomarker is selected from the group of CD55, CD271, MSCA-1 and CD146. Preferably, the biomarker is a protein biomarker. Preferably, the biomarker is a functionally active fragment of the protein biomarker. Preferably, the biomarker is a soluble variant of the protein biomarker. In preferred embodiments, the biomarker is associated with another molecule. For example, the protein biomarker may be at least two proteins joined together, for example in an aggregate, agglomerate or in a complex.

In the context of the invention, when referring to “sequence identity” or “identical sequence”, or “sequence identical to”, preferably this refers to a sequence identity of at least 60%, more preferably at least 70%, more preferably at least 80%, more preferably at least 85%, more preferably at least 90%, more preferably at least 95%, more preferably at least 97%, more preferably at least 98%, more preferably at least 99%, most preferably 100%.

Preferably, the biomarker comprises an amino acid sequence with a sequence identity of at least 60%, at least 70%, at least 80%, at least 90%, at least 95%, at least 97%, at least 98%, at least 99%, at least or 100% with an amino acid sequence corresponding to SEQ ID NOs. 1 to 14.

As used herein, the terms “identical” or percent “identity”, when used anywhere herein in the context of two or more nucleic acid or protein/polypeptide sequences, refer to two or more sequences or subsequences that are the same or have (or have at least) a specified percentage of amino acid residues or nucleotides that are the same (i.e., at, or at least, about 60% identity, preferably at, or at least, 65%, 70%, 75%, 80%, 85%, 90%, 91%, 92%, 93% or 94%, identity, and more preferably at, or at least, about 95%, 96%, 97%, 98%, 99%, or higher identity over a specified region-preferably over their full length sequences-, when compared and aligned for maximum correspondence over the comparison window or designated region) as measured using a sequence comparison algorithms, or by manual alignment and visual inspection (see, e.g., NCBI web site). In a particular embodiment, the percentage identity can be determined by the Blast searches; in particular for amino acid identity, those using BLASTP 2.2.28+ with the following parameters: Matrix: BLOSUM62; Gap Penalties: Existence: 11, Extension: 1; Neighboring words threshold: 11; Window for multiple hits: 40.

Preferably, the biomarker comprises an amino acid sequence with a sequence identity of at least 50%, at least 60%, at least 70%, at least 80%, at least 90%, at least 95%, at least 97%, at least 98%, at least 99%, at least or 100% with an amino acid sequence corresponding to SEQ ID NOs. 1 to 7. In these embodiments, the biomarker is CD55, also known as CR, DAF or Complement decay-accelerating factor. Said biomarker is referring to the Uniprot accession number P08174 and is at the time of the filing of the present application described by the uniprot entry found on https://www.uniprot.org/uniprotkb/P08174/entry. Preferably, the biomarker is selected from any of the 7 known isoforms, namely CD55 Isoform-1 (SEQ ID NO. 1, DAF-2), CD55 Isoform-2 (SEQ ID NO. 2, DAF-1), CD55 Isoform-3 (SEQ ID NO. 3, VDAF3), CD55 Isoform-4 (SEQ ID NO. 4, VDAF2), CD55 Isoform-5 (SEQ ID NO. 5, VDAF1), CD55 Isoform 6 (SEQ ID NO. 6, VDAF-4), and CD55 Isoform-7 (SEQ ID NO. 7, VDAF5).

Preferably, the biomarker comprises an amino acid sequence with a sequence identity of at least 50%, at least 60%, at least 70%, at least 80%, at least 90%, at least 95%, at least 97%, at least 98%, at least 99%, at least or 100% with an amino acid sequence corresponding to SEQ ID NO. 8 and 9. In these embodiments, the biomarker is CD271, also known as Gp80-LNGFR, Low affinity neurotrophin receptor p75NTR, Low-affinity nerve growth factor receptor (NGF receptor), p75 ICD or Tumor necrosis factor receptor superfamily member 16. Said biomarker is referring to the Uniprot accession number P08138 and is at the time of the filing of the present application described by the uniprot entry found on https://www.uniprot.org/uniprotkb/P08138/entry. Preferably, the biomarker is any of the two known isoforms, namely CD271 Isoform-1 (SEQ ID NO. 8) and CD271 Isoform-2 (SEQ ID NO. 9)

Preferably, the biomarker comprises an amino acid sequence with a sequence identity of at least 50%, at least 60%, at least 70%, at least 80%, at least 90%, at least 95%, at least 97%, at least 98%, at least 99%, at least or 100% with an amino acid sequence corresponding to SEQ ID NO. 10 and 11. In these embodiments, the biomarker is CD146, also known as Cell surface glycoprotein P1H12, Melanoma cell adhesion molecule, Melanoma-associated antigen A32, Melanoma-associated antigen MUC18, S-endo 1 endothelial-associated antigen, or Cell surface glycoprotein MUC18. Said biomarker is referring to the Uniprot accession number P43121 and is at the time of the filing of the present application described by the uniprot entry found on https://www.uniprot.org/uniprotkb/P43121/entry. Preferably, the biomarker is any of the two known isoforms, namely CD271 Isoform-1 (SEQ ID NO. 10) and CD271 Isoform-2 (SEQ ID NO. 11).

Preferably, the biomarker comprises an amino acid sequence with a sequence identity of at least 50%, at least 60%, at least 70%, at least 80%, at least 90%, at least 95%, at least 97%, at least 98%, at least 99%, at least or 100% with an amino acid sequence corresponding to SEQ ID NO. 12 to 14. In these embodiments, the biomarker is MSCA-1, also known as Tissue Non-Specific Alkaline Phosphatase, TNAP, TNSALP, Alkaline Phosphatase Biomineralization Associated, Alkaline Phosphatase Liver/Bone/Kidney Isozyme, Phosphoamidase, Phosphocreatine phosphatase. Said biomarker is referring to the Uniprot accession number P05186 and is at the time of the filing of the present application described by the uniprot entry found on https://www.uniprot.org/uniprotkb/P05186/entry. Preferably, the biomarker is any of the two known isoforms, namely MSCA-1 Isoform-1 (SEQ ID NO. 12), MSCA-1 Isoform-2 (SEQ ID NO. 13) and MSCA-1 Isoform-3 (SEQ ID NO. 14).

TABLE A
protein sequences.

| | |
|---|---|
| SEQ ID NO. 1 | MTVARPSVPAALPLLGELPRLLLLVLLCLPAVWGDC |
| CD55 | GLPPDVPNAQPALEGRTSFPEDTVITYKCEESFVKI |
| Canonical Isoform 1, | PGEKDSVICLKGSQWSDIEEFCNRSCEVPTRLNSA |
| DAF-2 | SLKQPYITQNYFPVGTVVEYECRPGYRREPSLSPK |
| Length: | LTCLQNLKWSTAVEFCKKKSCPNPGEIRNGQIDVP |
| 381 AA | GGILFGATISFSCNTGYKLFGSTSSFCLISGSSVQW |
| | SDPLPECREIYCPAPPQIDNGIIQGERDHYGYRQSV |
| | TYACNKGFTMIGEHSIYCTVNNDEGEWSGPPPEC |
| | RGKSLTSKVPPTVQKPTTVNVPTTEVSPTSQKTTT |
| | KTTTPNAQATRSTPVSRTTKHFHETTPNKGSGTTS |
| | GTTRLLSGHTCFTLTGLLGTLVTMGLLT |
| SEQ ID NO. 2 | MTVARPSVPAALPLLGELPRLLLLVLLCLPAVWGDC |
| CD55 | GLPPDVPNAQPALEGRTSFPEDTVITYKCEESFVKI |
| Isoform 2, DAF-1 | PGEKDSVICLKGSQWSDIEEFCNRSCEVPTRLNSA |
| Length: 440 AA | SLKQPYITQNYFPVGTVVEYECRPGYRREPSLSPK |
| | LTCLQNLKWSTAVEFCKKKSCPNPGEIRNGQIDVP |
| | GGILFGATISFSCNTGYKLFGSTSSFCLISGSSVQW |
| | SDPLPECREIYCPAPPQIDNGIIQGERDHYGYRQSV |
| | TYACNKGFTMIGEHSIYCTVNNDEGEWSGPPPEC |
| | RGKSLTSKVPPTVQKPTTVNVPTTEVSPTSQKTTT |
| | KTTTPNAQATRSTPVSRTTKHFHETTPNKGSGTTS |
| | GTTRLLSGSRPVTQAGMRWCDRSSLQSRTPGFK |
| | RSFHFSLPSSWYYRAHVFHVDRFAWDASNHGLAD |
| | LAKEELRRKYTQVYRLFLVS |
| SEQ ID NO. 3 | MTVARPSVPAALPLLGELPRLLLLVLLCLPAVWGDC |
| CD55 | GLPPDVPNAQPALEGRTSFPEDTVITYKCEESFVKI |
| Isoform 3, | PGEKDSVICLKGSQWSDIEEFCNRSCEVPTRLNSA |
| VDAF3 | SLKQPYITQNYFPVGTVVEYECRPGYRREPSLSPK |
| Length: 384 AA | LTCLQNLKWSTAVEFCKKKSCPNPGEIRNGQIDVP |
| | GGILFGATISFSCNTGYKLFGSTSSFCLISGSSVQW |
| | SDPLPECREIYCPAPPQIDNGIIQGERDHYGYRQSV |
| | TYACNKGFTMIGEHSIYCTVNNDEGEWSGPPPEC |
| | RGKSLTSKVPPTVQKPTTVNVPTTEVSPTSQKTTT |
| | KTTTPNAQATRSTPVSRTTKHFHETTPNKGSGTTS |
| | GTTRLLSALQVRPFEVSGSSHISSKKMMCIL |
| SEQ ID NO. 4 | MTVARPSVPAALPLLGELPRLLLLVLLCLPAVWGDC |
| CD55 | GLPPDVPNAQPALEGRTSFPEDTVITYKCEESFVKI |
| Isoform 4, | PGEKDSVICLKGSQWSDIEEFCNRSCEVPTRLNSA |
| VDAF2 | SLKQPYITQNYFPVGTVVEYECRPGYRREPSLSPK |
| Length: 364 AA | LTCLQNLKWSTAVEFCKKKSCPNPGEIRNGQIDVP |
| | GGILFGATISFSCNTGYKLFGSTSSFCLISGSSVQW |
| | SDPLPECREIYCPAPPQIDNGIIQGERDHYGYRQSV |
| | TYACNKGFTMIGEHSIYCTVNNDEGEWSGPPPEC |
| | RGKSLTSKVPPTVQKPTTVNVPTTEVSPTSQKTTT |
| | KTTTPNAQATRSTPVSRTTKHFHETTPNKGSGTTS |
| | GTTRLLSVLFM |
| SEQ ID NO. 5 | MTVARPSVPAALPLLGELPRLLLLVLLCLPAVWGDC |
| CD55 | GLPPDVPNAQPALEGRTSFPEDTVITYKCEESFVKI |
| Isoform 5, VDAF1 | PGEKDSVICLKGSQWSDIEEFCNRSCEVPTRLNSA |
| Length: 439 AA | SLKQPYITQNYFPVGTVVEYECRPGYRREPSLSPK |
| | LTCLQNLKWSTAVEFCKKKSCPNPGEIRNGQIDVP |
| | GGILFGATISFSCNTGYKLFGSTSSFCLISGSSVQW |
| | SDPLPECREIYCPAPPQIDNGIIQGERDHYGYRQSV |
| | TYACNKGFTMIGEHSIYCTVNNDEGEWSGPPPEC |
| | RGKSLTSKVPPTVQKPTTVNVPTTEVSPTSQKTTT |
| | KTTTPNAQATRSTPVSRTTKHFHETTPNKGSGTTS |
| | GTTRLLSETVFHRVIQDGLDLLASRSACLGLPKCW |
| | DYRREPPHLARAHVFHVDRFAWDASNHGLADLAK |
| | EELRRKYTQVYRLFLVS |
| SEQ ID NO. 6 | MTVARPSVPAALPLLGELPRLLLLVLLCLPAVWGDC |
| CD55 | GLPPDVPNAQPALEGRTSFPEDTVITYKCEESFVKI |
| Isoform 6, VDAF4 | PGEKDSVICLKGSQWSDIEEFCNRSCEVPTRLNSA |
| Length: 525 AA | SLKQPYITQNYFPVGTVVEYECRPGYRREPSLSPK |
| | LTCLQNLKWSTAVEFCKKKSCPNPGEIRNGQIDVP |
| | GGILFGATISFSCNTGYKLFGSTSSFCLISGSSVQW |
| | SDPLPECREIYCPAPPQIDNGIIQGERDHYGYRQSV |
| | TYACNKGFTMIGEHSIYCTVNNDEGEWSGPPPEC |
| | RGKSLTSKVPPTVQKPTTVNVPTTEVSPTSQKTTT |
| | KTTTPNAQGTETPSVLQKHTTENVSATRTPPTPQK |
| | PTTVNVPATIVTPTPQKPTTINVPATGVSSTPQRHTI |
| | VNVSATGTLPTLQKPTRANDSATKSPAAAQTSFISK |
| | TLSTKTPSAAQNPMMTNASATQATLTAQKFTTAKV |
| | AFTQSPSAAPTRSTPVSRTTKHFHETTPNKGSGTT |
| | SGTTRLLSGHTCFTLTGLLGTLVTMGLLT |
| SEQ ID NO. 7 | MTVARPSVPAALPLLGELPRLLLLVLLCLPAVWGDC |
| CD55 | GLPPDVPNAQPALEGRTSFPEDTVITYKCEESFVKI |
| Isoform 7, VDAF5 | PGEKDSVICLKGSQWSDIEEFCNRSCEVPTRLNSA |
| Length: 551 AA | SLKQPYITQNYFPVGTVVEYECRPGYRREPSLSPK |
| | LTCLQNLKWSTAVEFCKKKSCPNPGEIRNGQIDVP |
| | GGILFGATISFSCNTGYKLFGSTSSFCLISGSSVQW |
| | SDPLPECREIYCPAPPQIDNGIIQGERDHYGYRQSV |
| | TYACNKGFTMIGEHSIYCTVNNDEGEWSGPPPEC |
| | RGKSLTSKVPPTVQKPTTVNVPTTEVSPTSQKTTT |
| | KTTTPNAQGTETPSVLQKHTTENVSATRTPPTPQK |
| | PTTVNVPATIVTPTPQKPTTINVPATGVSSTPQRHTI |
| | VNVSATGTLPTLQKPTRANDSATKSPAAAQTSFISK |
| | TLSTKTPSAAQNPMMTNASATQATLTAQKFTTAKV |
| | AFTQSPSAARKSTNVHSPVTNGLKSTQRFPSAHIT |
| | ATRSTPVSRTTKHFHETTPNKGSGTTSGTTRLLSG |
| | HTCFTLTGLLGTLVTMGLLT |
| SEQ ID NO. 8 | MGAGATGRAMDGPRLLLLLLLGVSLGGAKEACPT |
| CD271 | GLYTHSGECCKACNLGEGVAQPCGANQTVCEPCL |
| Canonical isoform 1 | DSVTFSDVVSATEPCKPCTECVGLQSMSAPCVEA |
| Length: 427 AA | DDAVCRCAYGYYQDETTGRCEACRVCEAGSGLVF |
| | SCQDKQNTVCEECPDGTYSDEANHVDPCLPCTVC |
| | EDTERQLRECTRWADAECEEIPGRWITRSTPPEG |
| | SDSTAPSTQEPEAPPEQDLIASTVAGVVTTVMGSS |
| | QPVVTRGTTDNLIPVYCSILAAVVVGLVAYIAFKRW |
| | NSCKQNKQGANSRPVNQTPPPEGEKLHSDSGISV |
| | DSQSLHDQQPHTQTASGQALKGDGGLYSSLPPAK |
| | REEVEKLLNGSAGDTWRHLAGELGYQPEHIDSFT |
| | HEACPVRALLASWATQDSATLDALLAALRRIQRAD |
| | LVESLCSESTATSPV |
| SEQ ID NO. 9 | MSAPCVEADDAVCRCAYGYYQDETTGRCEACRV |
| CD271 | CEAGSGLVFSCQDKQNTVCEECPDGTYSDEANHV |
| Isoform 2 | DPCLPCTVCEDTERQLRECTRWADAECEEIPGRW |
| Length: 333 AA | ITRSTPPEGSDSTAPSTQEPEAPPEQDLIASTVAGV |
| | VTTVMGSSQPVVTRGTTDNLIPVYCSILAAVVVGLV |
| | AYIAFKRWNSCKQNKQGANSRPVNQTPPPEGEKL |
| | HSDSGISVDSQSLHDQQPHTQTASGQALKGDGGL |
| | YSSLPPAKREEVEKLLNGSAGDTWRHLAGELGYQ |
| | PEHIDSFTHEACPVRALLASWATQDSATLDALLAAL |
| | RRIQRADLVESLCSESTATSPV |
| SEQ ID NO. 10 | MGLPRLVCAFLLAACCCCPRVAGVPGEAEQPAPEL |
| CD146 | VEVEVGSTALLKCGLSQSQGNLSHVDWFSVHKEK |
| Canonical isoform 1 | RTLIFRVRQGQGQSEPGEYEQRLSLQDRGATLALT |
| Length: | QVTPQDERIFLCQGKRPRSQEYRIQLRVYKAPEEP |
| 646 AA | NIQVNPLGIPVNSKEPEEVATCVGRNGYPIPQVIWY |
| | KNGRPLKEEKNRVHIQSSQTVESSGLYTLQSILKA |
| | QLVKEDKDAQFYCELNYRLPSGNHMKESREVTVP |
| | VFYPTEKVWLEVEPVGMLKEGDRVEIRCLADGNP |
| | PPHFSISKQNPSTREAEEETTNDNGVLVLEPARKE |
| | HSGRYECQGLDLDTMISLLSEPQELLVNYVSDVRV |
| | SPAAPERQEGSSLTLTCEAESSQDLEFQWLREET |
| | GQVLERGPVLQLHDLKREAGGGYRCVASVPSIPG |
| | LNRTQLVNVAIFGPPWMAFKERKVWVKENMVLNL |
| | SCEASGHPRPTISWNVNGTASEQDQDPQRVLSTL |
| | NVLVTPELLETGVECTASNDLGKNTSILFLELVNLTT |
| | LTPDSNTTTGLSTSTASPHTRANSTSTERKLPEPES |
| | RGVVIVAVIVCILVLAVLGAVLYFLYKKGKLPCRRSG |
| | KQEITLPPSRKSELVVEVKSDKLPEEMGLLQGSSG |
| | DKRAPGDQGEKYIDLRH |
| SEQ ID NO. 11 | MVYIVRQFLLYNVSGSVYLDQLIVLLTAKFSILRIAGS |
| CD146 | RVHHSPFSGHLDGCSFLSLQHSLHTSLDMSRHEN |
| Isoform 2 | VFLGLTLSSKSAGLKGFQLAFVPGLLQGTGGYLDG |
| Length: 527 AA | PLPTPVDNPRVGLEVGLRLSLPPLPPCPGVHIQSS |
| | QTVESSGLYTLQSILKAQLVKEDKDAQFYCELNYRL |
| | PSGNHMKESREVTVPVFYPTEKVWLEVEPVGMLK |
| | EGDRVEIRCLADGNPPPHFSISKQNPSTREAEEET |
| | TNDNGVLVLEPARKEHSGRYECQGLDLDTMISLLS |
| | EPQELLVNYVSDVRVSPAAPERQEGSSLTLTCEAE |
| | SSQDLEFQWLREETGQVLERGPVLQLHDLKREAG |
| | GGYRCVASVPSIPGLNRTQLVNVAIFGPPWMAFKE |
| | RKVWVKENMVLNLSCEASGHPRPTISWNVNGTAS |
| | EQDQDPQRVLSTLNVLVTPELLETGVECTASNDLG |
| | KNTSILFLELVNLTTLTPDSNTTTGLSTSTASPHTRA |
| | NSTSTGKPGLAREQGCARASFLPCPSPESPVQKG |
| | E |
| SEQ ID NO. 12 | MISPFLVLAIGTCLTNSLVPEKEKDPKYWRDQAQET |
| MSCA-1 | LKYALELQKLNTNVAKNVIMFLGDGMGVSTVTAARI |
| Canonical isoform 1 | LKGQLHHNPGEETRLEMDKFPFVALSKTYNTNAQ |
| Length: 524 AA | VPDSAGTATAYLCGVKANEGTVGVSAATERSRCNT |
| | TQGNEVTSILRWAKDAGKSVGIVTTTRVNHATPSA |
| | AYAHSADRDWYSDNEMPPEALSQGCKDIAYQLMH |
| | NIRDIDVIMGGGRKYMYPKNKTDVEYESDEKARGT |
| | RLDGLDLVDTWKSFKPRYKHSHFIWNRTELLTLDP |
| | HNVDYLLGLFEPGDMQYELNRNNVTDPSLSEMVV |
| | VAIQILRKNPKGFFLLVEGGRIDHGHHEGKAKQALH |
| | EAVEMDRAIGQAGSLTSSEDTLTVVTADHSHVFTF |
| | GGYTPRGNSIFGLAPMLSDTDKKPFTAILYGNGPG |
| | YKVVGGERENVSMVDYAHNNYQAQSAVPLRHETH |
| | GGEDVAVFSKGPMAHLLHGVHEQNYVPHVMAYAA |
| | CIGANLGHCAPASSAGSLAAGPLLLALALYPLSVLF |
| SEQ ID NO. 13 | MPWSFRSSTPTWLRMSSCSWEMTYNTNAQVPDS |
| MSCA-1 | AGTATAYLCGVKANEGTVGVSAATERSRCNTTQG |
| Isoform 2 | NEVTSILRWAKDAGKSVGIVTTTRVNHATPSAAYAH |
| Length: 447 AA | SADRDWYSDNEMPPEALSQGCKDIAYQLMHNIRDI |
| | DVIMGGGRKYMYPKNKTDVEYESDEKARGTRLDG |
| | LDLVDTWKSFKPRYKHSHFIWNRTELLTLDPHNVD |
| | YLLGLFEPGDMQYELNRNNVTDPSLSEMVVVAIQIL |
| | RKNPKGFFLLVEGGRIDHGHHEGKAKQALHEAVE |
| | MDRAIGQAGSLTSSEDTLTVVTADHSHVFTFGGYT |
| | PRGNSIFGLAPMLSDTDKKPFTAILYGNGPGYKVV |
| | GGERENVSMVDYAHNNYQAQSAVPLRHETHGGE |
| | DVAVFSKGPMAHLLHGVHEQNYVPHVMAYAACIG |
| | ANLGHCAPASSAGSLAAGPLLLALALYPLSVLF |
| SEQ ID NO. 14 | MFLGDGMGVSTVTAARILKGQLHHNPGEETRLEM |
| MSCA-1 | DKFPFVALSKTYNTNAQVPDSAGTATAYLCGVKAN |
| Isoform 3 | EGTVGVSAATERSRCNTTQGNEVTSILRWAKDAG |
| Length: 469 AA | KSVGIVTTTRVNHATPSAAYAHSADRDWYSDNEM |
| | PPEALSQGCKDIAYQLMHNIRDIDVIMGGGRKYMY |
| | PKNKTDVEYESDEKARGTRLDGLDLVDTWKSFKP |
| | RYKHSHFIWNRTELLTLDPHNVDYLLGLFEPGDMQ |
| | YELNRNNVTDPSLSEMVVVAIQILRKNPKGFFLLVE |
| | GGRIDHGHHEGKAKQALHEAVEMDRAIGQAGSLT |
| | SSEDTLTVVTADHSHVFTFGGYTPRGNSIFGLAPM |
| | LSDTDKKPFTAILYGNGPGYKVVGGERENVSMVDY |
| | AHNNYQAQSAVPLRHETHGGEDVAVFSKGPMAHL |
| | LHGVHEQNYVPHVMAYAACIGANLGHCAPASSAG |
| | SLAAGPLLLALALYPLSVLF |

In preferred embodiments, detection of the presence and/or the expression comprises detecting a measurement reading from a sample derived from a potentially immunomodulatory MSC, such as the emission and/or absorption of light, the binding of a molecule, an enzymatic reaction, wherein the measurement reading is indicative for the presence of the biomarker.

In preferred embodiments, the method comprises the detection of the presence of two, preferably three, more preferably four biomarkers selected from the group CD55, CD271, MSCA-1 and CD146 associated with the MSC, wherein the presence and/or expression of the two, preferably the three, more preferably four biomarkers is indicative for an immunomodulatory activity of the MSC.

Generally, detection of the biomarker associated with the MSC does not prerequisite that the biomarker needs to be associated with the MSC at the moment, when the detection method is conducted. For example, the invention also includes the option to isolate the biomarker from the MSCs and to detect the biomarker that is isolated from the MSC.

In preferred embodiments, the biomarker is associated with an MSC. Preferably, the biomarker associated with an MSC is attached to the surface of the MSC, is situated within the MSC and/or is released from the MSC. Preferably, the term “released by the MSC” refers to a secretion of the biomarker from the MSC. Preferably, released refers to a destruction of the MSC, whereby the biomarker is released in the environment surrounding the MSC. In preferred embodiments, the biomarker is a surface marker. In such an embodiment, the biomarker may be attached to the surface of the MSC and comprises at least one domain that is on the exterior of the MSCs. Preferably, the MSC surface marker may be released from the MSC.

In preferred embodiments, the immunomodulatory activity of the MSC is characterized by at least one of:
(i) a differentiation, activation and/or proliferation of cells associated with the innate or adaptive immune system, preferably wherein the cells are MNCs such as PBMNCs, or selected immune cell subsets;
(ii) a presence and/or expression of an immunomodulating factor;
(iii) not requiring the complement system to function.

The term “immunomodulatory activity” in the context of the present invention refers to a stimulation, an activation, a suppression or an inhibition, either complete or partially, of an immune response of an organism and/or in vitro. In this context, the term “immune response” may refer to a differentiation, activation and/or proliferation of cells associated with the innate or adaptive immune system (for example PBMNCs, or selected immune cell subsets), the release of compounds that have a regulatory function to the immune system, or the generation, or release of antigen-recognizing molecules such as antibodies, T-cell receptors or fragments thereof. Preferably, the immunomodulatory activity is exerted by an interaction between the MSC and another cell, preferably an immune cell. In preferred embodiments, the interaction may be cell-to-cell contact between an MSC and the another cell and/or by a compound, such as the immunomodulatory factor, released from the MSC. In preferred embodiments, the immunomodulatory activity of the MSC is an immunosuppressive or immunostimulatory activity.

Preferably, the expression of the immunomodulating factor is an upregulation of the immunomodulating factor. Preferably, the immunomodulating factor is expressed on the surface of the MSCs or is secreted into the environment surrounding the MSCs.

In preferred embodiments, the immunomodulating factor is selected from the group of CXCL9, IDO1, PTGS2, HLA-DRB1, CCL5, CXCL5, TNFAIP6, CD59, PTGES3L-AARSD1, IL6ST, IFNGR1, PTGDS, HPGDS, CD274, CD38, VCAM1, TICAM1, PDPN, THY1, and ADORA2A.

Preferably, the another cell is a cell of the adaptive or native immune system. Preferably, the another cell is a monocyte (such as macrophage), dendritic cell, B cell, or T cell.

In preferred embodiments, the immunomodulatory factor is selected from the group of HLA-DR, CD274, IDO-1, PGE2, preferably wherein the immunomodulating factor is IDO-1.

In preferred embodiments, the presence refers to a quantity. For example, the quantity may be a concentration, an amount, such as an absolute or normalized amount.

In preferred embodiments, the presence and/or the expression of the biomarker, and optionally of the immunomodulating factor, is compared to the presence and/or expression of a reference, preferably wherein the reference is a reference value or a reference sample.

In this context, a reference value is to be understood as a predetermined presence of the at least one biomarker and/or a measurement reading. Such a reference value may indicate an immunomodulatory activity of a sample. When comparing the presence of the at least one biomarker and/or the measurement reading of a sample derived from a potentially immunomodulatory MSC to the reference value, a higher or lower presence of the at least one biomarker and/or measurement reading may indicate an immunomodulatory activity of the mesenchymal stromal cell.

In this context, a reference sample is to be understood as a MSC with a predetermined immunomodulatory activity, a composition comprising a, preferably predetermined, amount of MSC with a predetermined immunomodulatory activity, or a composition comprising the at least one biomarker of the present invention with a predetermined immunomodulatory activity. Preferably, a measurement reading indicative for the presence of the biomarker, for example the emission and/or absorption of light, the binding of a molecule, an enzymatic reaction of the reference sample is detected. Preferably, the measurement reading is compared to the measurement reading of a sample derived from a potentially immunomodulatory active MSC, wherein a higher or lower presence of the at least one biomarker and/or measurement reading may indicate an immunomodulatory activity of the mesenchymal stromal cell.

In preferred embodiments, the reference has a predetermined immunomodulatory activity, preferably wherein the predetermined immunomodulatory activity is one or more of the following: an absence of an immunomodulatory activity, the presence and/or expression of a biomarker, the presence and/or expression of an immunomodulating factor, an inhibition of PBMNC proliferation or selected immune cell subset proliferation, and/or the presence or absence of a measurement reading.

In preferred embodiments, the presence and/or expression of the biomarker is detected in the proteome and/or transcriptome of the MSC. In this context, the detection in the proteome preferably refers to the detection of the presence of the biomarker protein. In this context, the detection in the transcriptome preferably refers to the detection of the presence of the biomarker by detecting the presence of a nucleic acid encoding for the biomarker.

Preferably, the presence and/or expression of the biomarker is detected using a detection method selected from spectroscopy, mass spectrometry, flow cytometry, and/or a biological assay.

Preferably, the mass spectrometry refers to a method comprising matrix-assisted laser desorption/ionization (MALDI) and/or electrospray ionization (ESI).

Preferably, the mass spectrometry comprises a method selected from the group of gas chromatography-mass spectrometry (GC-MS), liquid chromatography-mass spectrometry (LC-MS), ion mobility spectrometry (IMS-MS), and/or capillary electrophoresis-mass spectrometry (CE-MS). Preferably, the mass spectrometry comprises no labelling of the MSC. In other preferred embodiments, the mass spectrometry may comprise a step of labelling the biomarker and/or the MSC, such as labelling with an isobaric tag, 180 label, dimethyl label and/or isotope-coded affinity label.

Preferably, the spectroscopy may be selected from the group of UV/Vis spectroscopy, fluorescence spectroscopy, Nuclear magnetic resonance (NMR)-spectroscopy, and/or Infrared (IR)-spectroscopy, Raman-spectroscopy and/or surface plasmon resonance spectroscopy. In preferred embodiment, the spectroscopy includes a labelling step of the biomarker and/or the MSC. For example, such a label may be a fluorescent label.

Preferably, the biological assay is an immunoassay. A person skilled in the art will be familiar with various such immunoassay methods. Any immunological/biological/in vitro assay that is suitable for detecting a biomarker according to the present invention is included herein. Preferably, the immunoassay method is an enzyme-linked immunosorbent assay (ELISA), enzyme immunoassay (EIA), fluorescence immunoassay (FIA), Chemiluminescence immunoassay (CIA), radioimmunoassay (RIA), Western-Blot and/or Peptide-Array. Preferably, the assay comprises a binding agent that binds to the biomarker. In preferred embodiments, the binding agent may be attached on a surface, such as the surface of a microscopy slide or the surface of a well plate. Preferably, the binding-agent provides a detectable signal, such as a tag comprising a fluorescent moiety. In preferred embodiments, a first binding agent fixates the biomarker and, if the biomarker is attached to the MSC, may also fixate the MSC. Preferably, in such embodiments, an additional binding agent may be used that provides a detectable signal and preferably binds to the MSC, to the biomarker, and/or to the binding agent that binds to the first biomarker. Preferably, the first binding agent or a tag thereof is an intermediate or precursor the additional binding agent to specifically bind to, for example such as in the case of a biotin tagging system or enzyme tagging systems. Preferred, the tag of the binding agent is a catalytic tag such as horseradish peroxidase, alkaline phosphatase, glucose oxidase and β-galactosidase.

Preferably, the detection of the presence of a nucleic acid may comprise conducting a method selected from the group of PCR (such as quantitative PCR, also known as qPCR), Northern-Blot, RNase Protection Assay, DNA-microarray, serial analysis of gene expression (SAGE), differential display, RNA-Sequencing, and ribosomal profiling. Preferably, detection of the presence of the biomarker in the form of a nucleic acid may comprise next generation sequencing (NGS).

In preferred embodiments, the methods disclosed herein the detection method may be a combination of any detection method herein. Furthermore included, are any variants of the detection methods herein.

In the context of the present invention, the term “mesenchymal stromal cell” refers to a multipotent stromal cell that can differentiate into a variety of cell types, including, but not limited to osteoblasts (bone cells), chondrocytes (cartilage cells), myocytes (muscle cells) and adipocytes (fat cells). These cells are also known as “mesenchymal stem cells” due to their multi-potency. Preferred MSCs of the present invention are able to support haematopoiesis, can differentiate along mesenchymal and non-mesenchymal lineages in vitro, are capable of suppressing alloresponses and appear to be non-immunogenic.

MSC are, in particular in context of the present invention, useful due to their regenerative function by paracrine or endocrine mechanisms, their immune-suppressive activity as well as their ability to form direct cell-to-cell interactions. These cells are known to either secrete certain proteins and/or compounds, which is in turn influence the microenvironment around the cells, or when not directly expressing the proteins themselves, mesenchymal stromal cells are known to influence the downstream expression of other proteins, which for example, may be affected by the presence or absence of a mesenchymal stromal cell.

That is to say that the presence of a mesenchymal stromal cell within the environment of another cell can cause the other cell to start producing, or even secreting, certain proteins. Therefore, disclosed herein are both compounds and/or proteins secreted by the mesenchymal stromal cells themselves and are therefore considered to be derived from MSCs as they are specifically obtained from MSCs, as well as proteins and compounds that are produced and/or secreted as a result of the presence of mesenchymal stromal cells within the cell environment. Included therein is also the downstream expression of proteins that is initiated via one or more expression cascades due to the presence of mesenchymal stromal cells in the microenvironment. MSC according to the invention may be obtained from a variety of sources such as bone marrow, adipose tissue, umbilical cord blood and others.

Preferably, the MSC is a bone marrow, adipose tissue, peripheral blood, umbilical cord blood, embryonic tissue, fetal blood, fetal heart, amnion fluid, amniotic membrane, placenta, synovia, trabecular bone, periosteum, tendon, dermis, teeth, muscle, brain, thymus, kidney, liver, fetal liver, fetal pancreas, pancreas, spleen, lung-derived MSC. In preferred embodiments, the MSC is a bone marrow MSC. Preferably, the animal is mammal. Preferably, the mammal is a human, monkey such as a rhesus monkey, horse, sheep, pig, cattle, dog, cat, rabbit, mouse and/or rat. Preferably, the mammal is a human. In preferred embodiments, the MSC is a human bone marrow MSC.

In preferred embodiments, the MSC is cultured in a non-inflammatory environment prior to the detection of the presence of the biomarker, and optionally the detection of the presence and/or expression of the immunomodulating factor, preferably wherein the biomarker is CD55 and/or CD146, preferably CD55.

In preferred embodiments, the MSC is exposed to and/or cultured in an inflammatory environment prior to the detection of the presence and/or expression of the biomarker, and optionally the detection of the presence and/or expression of the immunomodulating factor, preferably wherein the biomarker is CD271 and/or MSCA-1.

The inflammatory environment may comprise a co-culture of the MSC and at least one allogeneic activated MNC, such an allogeneic PBMNC, or a selected immune cell subset, including a human allogeneic activated PBMNC (hPBMNC). In this context, a co-culture may refer to a culture, wherein the MSC is cultured in a shared medium that comprises at least one allogeneic activated MNC, such an allogeneic PBMNC including a human allogeneic activated PBMNC (hPBMNC). Preferably, the co-culture of the MSC may refer to a culture, wherein the MSC is cultured in the shared medium that comprises at least one allogeneic activated MNC and the MSC is contacted with the at least one allogeneic activated MNC. Preferably, the co-culture of the MSC may refer to a culture, wherein the MSC is cultured in the shared medium that comprises at least one allogeneic activated MNC and the MSC is not contacted with the at least one allogeneic activated MNC, for example by culturing the cells in separate compartments that share the same medium but do not allow for an exchange of cells. Preferably, the inflammatory environment may be a medium comprising a compound associated with an allogeneic activated MNC, for example a compound secreted by an allogeneic activated MNC, and/or the inflammatory environment may be a medium comprising the compound associated with an allogeneic activated MNC. Preferably, the inflammatory environment may be a medium, in which at least one allogeneic activated MNC was cultured in. Preferably, the inflammatory environment may be a medium comprising proinflammatory cytokines for example HIF1α (hypoxia-inducible factor 1α), IFNγ, TNFα, TGF β, IL-1 and/or Toll like receptor (TLR) agonists. Preferably, the inflammatory environment may be a hypoxic medium.

In a second aspect, the invention pertains to a method for the manufacture of an immunomodulatory preparation derived from an MSC with immunomodulatory activity.

In preferred embodiments, said method comprises determining a presence or absence of an immunomodulatory activity of an MSC according to the first aspect of the invention.

In preferred embodiments, the method for the manufacture of an immunomodulatory preparation derived from an MSC with immunomodulatory activity comprises the steps of providing a cellular preparation comprising an MSC, determining a presence or absence of an immunomodulatory activity of an MSC in the cellular preparation comprising an MSC, wherein, in the case that the presence of the immunomodulatory activity of an MSC in the cellular preparation is determined, the immunomodulatory preparation derived from an MSC with immunomodulatory activity is obtained.

In preferred embodiments, the method for the manufacture of an immunomodulatory preparation derived from an MSC with immunomodulatory activity comprises at least one additional manufacturing step after the step of determining the presence or absence of an immunomodulatory activity of the MSC in the cellular preparation comprising an MSC.

In preferred embodiments, the method for the manufacture of an immunomodulatory preparation derived from an MSC with immunomodulatory activity comprises a culturing of the MSC. In preferred embodiments, the culturing of the MSC is conducted in an inflammatory environment. Preferably, the inflammatory environment comprises a co-culture of the MSC and an allogeneic activated MNC, such an allogeneic activated PBMNC.

In preferred embodiments, the method for the manufacture of an immunomodulatory preparation derived from an MSC with immunomodulatory activity comprises the recombinant expression of a biomarker selected from the group of CD55, CD271, MSCA-1 and CD146.

In preferred embodiments, the method for the manufacture of an immunomodulatory preparation derived from an MSC with immunomodulatory activity comprises a step of isolating an MSC, preferably the MSC with immunomodulatory activity, a vesicle, a micelle and/or a compound, wherein the vesicle, micelle, and/or compound are associated with the MSC with immunomodulatory activity or are associated with a cell that is exposed to the MSC with immunomodulatory activity, exposed to a micelle associated with the MSC with immunomodulatory activity, exposed to a vesicle associated with the MSC with immunomodulatory activity and/or the compound associated with the MSC with immunomodulatory activity.

In preferred embodiments, the method for the manufacture of an immunomodulatory preparation derived from an MSC with immunomodulatory activity, comprises isolating a cell that is exposed to the MSC with immunomodulatory activity, exposed to a micelle associated with the MSC with immunomodulatory activity, exposed to a vesicle associated with the MSC with immunomodulatory activity and/or the compound associated with the MSC with immunomodulatory activity.

In preferred embodiments, the method for the manufacture of an immunomodulatory preparation derived from an MSC with immunomodulatory activity the vesicle is an extracellular vesicle.

In preferred embodiments, the method for the manufacture of an immunomodulatory preparation derived from an MSC with immunomodulatory activity the compound is a biomarker selected from CD55, CD146, MSCA-1 and/or CD271

In preferred embodiments, the method for the manufacture of an immunomodulatory preparation derived from an MSC with immunomodulatory activity the compound is an immunomodulatory factor according to the first aspect of the invention.

In preferred embodiments, the method for the manufacture of an immunomodulatory preparation derived from an MSC with immunomodulatory activity, the step of isolating comprises isolating the MSC, preferably the MSC with immunomodulatory activity, the vesicle associated with the MSC with immunomodulatory activity, the micelle associated with the MSC with immunomodulatory activity and/or the compound associated with the MSC with immunomodulatory activity from an organism, preferably wherein the organism is a mammal.

In a third aspect, the invention pertains to an immunomodulatory preparation derived from an MSC with immunomodulatory activity, wherein the preparation comprises:
(i) the MSC with immunomodulatory activity,
(ii) a cell, vesicle, a micelle and/or a compound according to any aspect of the invention, and/or
(iii) a medium from a culture of the MSC with immunomodulatory activity.

In preferred embodiments, the immunomodulatory preparation derived from an MSC with immunomodulatory activity is obtainable by the method according to the second aspect of the invention.

In preferred embodiments the immunomodulatory preparation derived from an MSC with immunomodulatory activity is characterized by a presence and/or expression, preferably an increased presence and/or expression, of a biomarker selected from the group of CD55, CD146, MSCA-1 and CD271.

In preferred embodiments, the immunomodulatory preparation derived from an MSC with immunomodulatory activity, comprises one or more MSC subpopulations selected from the group of:

$$\begin{matrix}a ) & \\ MSCA 1 - CD 55 - CD 146 + CD 119 + CD 130 + PDPN - C D 140 a + CD 106 - CD 271 - & \end{matrix}$$

$$\begin{matrix}b ) & \\ MSCA 1 - CD 55 - CD 146 + CD 119 + CD1 \; 30 + PDPN - C D 140 a + C D 106 - CD 271 + & \end{matrix}$$

$$\begin{matrix}c ) & \\ MSCA 1 - CD 55 - CD 146 + CD 119 + CD 130 + PDPN - C D 140 a + CD 106 + CD 271 - & \end{matrix}$$

$$\begin{matrix}d ) & \\ MSCA 1 - CD 55 - CD 146 + CD 119 + CD 130 + PDPN + CD 140 a + CD 106 - CD 271 - & \end{matrix}$$

$$\begin{matrix}e ) & \\ MSCA 1 - CD 55 - CD 146 + CD 119 + CD 130 - PDPN - CD 140 a + CD 106 - CD 271 - & \end{matrix}$$

$$\begin{matrix}f ) & \\ MSCA 1 - CD 55 - CD 146 + CD 119 + CD 130 - PDPN - CD 140 a + CD 106 - CD 271 + & \end{matrix}$$

$$\begin{matrix}g ) & \\ MSCA 1 - CD 55 + CD 146 + CD 119 + CD 130 + PDPN - CD 140 a + CD 106 - CD 271 - & \end{matrix}$$

$$\begin{matrix}h ) & \\ MSCA 1 - CD 55 + CD 146 + CD 119 + CD 130 + PDPN + CD 140 a + CD 106 - CD 271 - & \end{matrix}$$

$$\begin{matrix}i ) & \\ MSCA 1 - CD 55 + CD 146 + CD 119 + CD 130 + PDPN + CD 140 a + CD 106 + CD 271 - & \end{matrix}$$

$$\begin{matrix}j ) & \\ MSCA 1 - CD 55 + CD 146 + CD 119 + CD 130 - PDPN + CD 140 a + CD 106 + CD 271 - & \end{matrix}$$

In preferred embodiments of the invention the one or more MSC subpopulations is an immunomodulatory MSC subpopulation.

In a fourth aspect, the invention pertains to an immunomodulatory MSC preparation according to any aspect of the invention, for use in the treatment of a condition or disease.

In preferred embodiments of the invention, the disease is selected from the group of an immune disease, preferably an autoimmune disease, a graft-versus-host disease, an inflammatory disease, neurodegenerative disease and/or tissue damage. In preferred embodiments of the invention, the disease is selected from the group of a liver disease, heart disease, bone/cartilage disease, spinal cord injury, brain disease and/or lung disease.

Preferably the inflammatory disease is an autoimmune disease. Preferably, the inflammatory disease is selected from the group of allergy, asthma, Alzheimer's disease, autoimmune diseases, autoimmune encephalomyelitis, autoimmune glomerular diseases, autoimmune thyroid disease, autoimmune lymphoproliferative disease, cancer, coeliac disease, Crohn's disease, demyelinating disease, endometriosis, fatty liver disease, glomerulonephritis, hepatitis, Graves' disease, multiple sclerosis, inflammatory bowel disease, lupus, immune gastritis, Parkinson's disease, reperfusion injury, rheumatoid arthritis, scleroderma, type 1 diabetes mellitus, type 2 diabetes mellitus, transplant rejection, uveitis.

Preferably neurodegenerative disease is selected from a disease from the group above Alzheimer's disease, Parkinson's disease, stroke, and brain cell injuries.

In preferred embodiments of the invention, the condition or disease is an immune disease.

In preferred embodiments of the invention, the disease is celiac disease, diabetes mellitus type 1, Graves' disease, inflammatory bowel disease, multiple sclerosis, psoriasis, rheumatoid arthritis, systemic lupus erythematosus and/or graft-versus-host disease.

Preferably, the treatment is a regenerative treatment, preferably, wherein the regenerative treatment is a regeneration of tissue, such as bone, cartilage, muscle, ligament, tendon, skin and adipose tissues.

In a fifth aspect, the invention pertains to a method for identifying an immunomodulatory population of MSCs with a known presence and/or expression of a biomarker, comprising:
(i) co-culturing an MSC population, and allogeneic activated PBMNCs in separate or joint compartments with shared media and
(ii) detecting a presence and/or expression of a biomarker of the co-cultured MSC population, preferably by transcriptome analysis and/or proteome analysis and comparing said presence and/or expression of the biomarker of the co-cultured MSC population with the presence and/or expression of the biomarker mono-cultured MSCs of the MSC population; and
(iii) detecting a presence and/or expression of an immunomodulating factor in the co-culture, preferably by transcriptome analysis and/or proteome analysis and comparing said presence and/or expression of immunomodulating factor in the co-culture with the presence and/or expression of the immunomodulating factor in a culture of mono-cultured MSCs of the MSC population; and/or
(iv) detecting the proliferation inhibition of the co-cultured allogeneic activated PBMNCs, or selected immune cell subsets, and comparing said proliferation inhibition with the proliferation of allogeneic activated PBMNCs, or selected immune cell subsets, that are cultured in a separate culture that does not share media with the MSC population;
   wherein an increased presence of the immunomodulating factor, an increased expression of the immunomodulating factor and/or an increased proliferation inhibition of the allogeneic activated PBMNCs indicates an immunomodulatory MSC population; and optionally,
(v) phenotyping the MSC population regarding the presence and/or expression of said biomarker, thus resulting in the identification of immunomodulatory population of MSCs with a known presence and/or expression of a biomarker.

Preferably, in the method for identifying an immunomodulatory population of MSCs with a known presence and/or expression of a biomarker, at least two of an increased presence of the immunomodulating factor, an increased expression of the immunomodulating factor and an increased proliferation inhibition of the allogeneic activated PBMNCs, or selected immune cell subsets, indicates an immunomodulatory MSC population or stage. Preferably, an increased presence of the immunomodulating factor, an increased expression of the immunomodulating factor and an increased proliferation inhibition of the allogeneic activated PBMNCs, or selected immune cell subsets, indicates an immunomodulatory MSC population.

Preferably, the immunomodulatory MSC population with a known presence and/or expression of a biomarker that is identified exerts its immunomodulatory activity by a mechanism, such as by cell-to-cell interaction and/or by the secretion of immunomodulatory compounds. Preferably, the method indicates, if the immunomodulatory activity is MNC (for example PBMNC) mediated.

Preferably, the detecting a presence and/or expression of a biomarker in this aspect comprises detecting the presence and/or expression of the biomarker in the proteome and/or transcriptome of the MSC and/or the sample comprising the MSC. Preferably, the presence and/or expression of the biomarker is detected using a detection method according to the first aspect of the invention.

Preferably, the detecting a presence and/or expression of an immunomodulating factor in this aspect comprises detecting the presence and/or expression of the biomarker in the proteome and/or transcriptome of the co-culture. Preferably, the presence and/or expression of the immunomodulating factor is detected using a detection method that may be used for the detection for the presence or the expression of a biomarker according to the first aspect of the invention.

In preferred embodiments, the MSC population originates from a sample containing MSCs only of an individual donor. In such embodiments, while MSC population originates from a sample containing MSCs only of an individual donor, the sample may comprise MSC populations with varying immunomodulatory activity. For example, the immunomodulatory activity between the MSC populations in such a sample may vary depending on the tissue type of the sample. Furthermore, the immunomodulatory activity of MSCs from a sample of an individual donor may also for vary, if the MSCs are from the same tissue type. Therefore, the present method may be used to identify an immunomodulatory MSC population from MSCs originating from such a sample.

In other preferred embodiment, the method may be used to identify a donor that comprises an immunomodulatory MSC population.

In preferred embodiments, the biomarker is a MSC subpopulation, or wherein the biomarker is selected from the group of CD55, CD271, MSCA-1 and CD146. Preferably, the biomarker is selected from the group of CD55, CD271, MSCA-1 and CD146. In embodiments, wherein the biomarker is selected from the group of CD55, CD271, MSCA-1 and CD146, the biomarker may be defined according to the first aspect of the invention. Preferably, the biomarker is a MSC subpopulation. Preferably, the MSC subpopulation comprises a biomarker, preferably wherein the biomarker is selected from the group of CD55, CD271, MSCA-1 and CD146. In the embodiments, wherein the biomarker is a MSC subpopulation, the immunomodulatory population of MSCs may be a MSCs population comprising the MSC subpopulation. For example, this may refer to a population of MSCs, wherein immunomodulatory and non-immunomodulatory MSCs are present. By detecting the presence of the biomarker “MSC subpopulation”, such a MSC population may then be identified as an “immunomodulatory population of MSCs”, by performing the rest of the steps of the method for identifying an immunomodulatory population of MSCs with a known presence and/or expression of a biomarker.

In preferred embodiments, the presence and/or expression refers to a quantity. For example, the quantity may be a concentration, an amount, such as an absolute or normalized amount.

In preferred embodiments, the method comprises phenotyping a MSC subpopulation in the MSC population. A population of MSCs may comprise multiple MSC subpopulations. For example, a sample of an individual donor may comprise MSC subpopulations with varying immunomodulatory activity. By phenotyping a MSC subpopulation in the MSC population, a phenotype profile of the immunomodulatory MSC population may be provided. Preferably, the presence and/or expression of a biomarker of the immunomodulatory population of MSCs with a known presence and/or expression of a biomarker is such a phenotype profile.

In preferred embodiments, the method comprises an additional step of functional validation of an immunomodulatory activity of the population of MSCs.

In preferred embodiments, the additional step of functional validation comprises culturing of a population of MSCs depleted or enriched of the biomarker and one or both of the following:
(i) detecting the presence and/or expression of the immunomodulating factor, in a cell lysate of the population of MSCs depleted or enriched of the biomarker and/or in the cell culture medium of the population of MSCs depleted or enriched of the biomarker;
(ii) detecting PBMNC proliferation inhibition, wherein PBMNCs, or selected immune cell subsets, are co-cultured with the population of MSCs depleted or enriched of the biomarker, preferably in the same compartment;
   wherein the presence and/or expression of the immunomodulating factor and/or the PBMNC proliferation inhibition are compared to the presence and/or expression of the immunomodulating factor and/or the PBMNC proliferation inhibition of a reference, preferably wherein the reference is a population of wild-type MSCs.

In preferred embodiments, the immunomodulating factor is an immunomodulating factor according to step (iii) of the present aspect and/or the immunomodulating factor is an immunomodulating factor according to the first aspect of the invention. Preferably, the immunomodulating factor is IDO-1.

In preferred embodiments, the method comprises phenotyping a MSC subpopulation in the MSC population.

In preferred embodiments, the presence and/or expression of the biomarker is correlated to the presence and/or expression of the immunomodulating factor.

In the context of the invention, the expression MSC “population” or “subpopulation” preferably refers to stages that the MSCs are in. For example, when referring to an “immunomodulatory population” or “immunomodulatory subpopulation”, this may refer to respective MSC stages.

In a sixth aspect, the invention pertains to a method for identifying an immunomodulatory subpopulation of MSCs with a known presence and/or expression of a biomarker, wherein the method is the method according to any one of the previous claims, wherein the immunomodulatory population of MSCs is an immunomodulatory subpopulation of MSCs and wherein the method comprises an additional step of functional validation of an immunomodulatory activity of a subpopulation of MSCs depleted of the biomarker.

In preferred embodiments of the invention the additional step of functional validation comprises culturing of the subpopulation of MSCs depleted of the biomarker and one or both of the following:
(i) Detecting the presence and/or expression of the immunomodulating factor, preferably IDO-1, in a cell lysate of the subpopulation of MSCs depleted of the biomarker and/or in the cell culture medium of the subpopulation of MSCs depleted of the biomarker;
(ii) detecting PBMNC, or selected immune cell subsets, proliferation inhibition, wherein PBMNCs are co-cultured with the subpopulation of MSCs depleted of the biomarker, preferably in the same compartment;
wherein the presence and/or expression of the immunomodulating factor and/or the PBMNC proliferation inhibition are compared to presence and/or expression of the immunomodulating factor and/or the PBMNC proliferation inhibition of a reference, preferably wherein the reference is a population of wild-type MSCs.

In a seventh aspect, the invention pertains to an agent for determining a presence or absence of an immunomodulatory activity of an MSC, wherein the agent specifically binds to a biomarker selected from the group of CD55, CD271, MSCA-1 and CD146.

In a preferred embodiment, the presence of the agent and/or its binding to the biomarker is detectable using a detection method selected from spectroscopy, mass spectrometry, flow cytometry and/or a biological assay.

In preferred embodiments of the invention, the agent is a ligand molecule, an antigen binding construct or a derivative thereof specific for the biomarker, preferably wherein the antigen binding construct is a protein or nucleic acid, preferably an antibody or antigen binding fragment, a T-cell receptor, an aptamer, a nanobody, or other molecular antigen binding construct or derivative thereof specifically binding to the biomarker.

Further provided is in view of the aforesaid, a use of a method of the first aspect in a quality control of an MSC preparation, or of a preparation of EV obtained from MSCs. Preferably, the at least one biomarker is CD55. Such use may involve detecting the presence of absence of CD55 presence and/or expression in a preexisting preparation of MSCs or pre-existing preparation of EV.

Preferably the use involves detecting the presence and/or expression of CD55 using any one or a combination of the following methods: polymerase chain reaction (PCR), flow cytometry such as fluorescent activated cytometry or cell sorting (FACS), or by using one or more anti-CD55 antibodies, for example in a Western Blot.

Further provided is in view of the aforesaid, a use of a method for enriching an immunologically active MSC preparation or EV preparation, the method comprising providing a previously obtained MSC population or and/or EV preparation (obtained from MSC),
(i) performing a method according to for example the first aspect with MSCs in the previously obtained MSC or EVs;
(ii) obtaining an enriched immunologically active MSC preparation or EV preparation by combining only such MSC or EVs into a new MSC preparation or EV preparation for which a presence and/or an expression of the at least one biomarker is detected, and optionally discarding any MSC or EV for which no presence and/or an expression of the at least one biomarker is detected.

Most preferred is that the at least one biomarker is CD55. Preferably, the presence and/or an expression of the at least one biomarker is detected compared to a reference value, preferably wherein the reference value is a negative or positive control reference.

Further provided is in view of the aforesaid, a method of producing a genetically modified MSC, the method comprising a step of introducing into the MSC an expressible sequence encoding a recombinant CD55 protein, optionally expressing the recombinant CD55 protein in the genetically modified MSC.

Further provided is in view of the aforesaid, a genetically modified MSC or an EV obtained therefrom, wherein the genetically modified MSC or EV obtained therefrom comprises an recombinantly expressed CD55 protein, preferably wherein the recombinantly expressed CD55 is expressed by a genetically introduced recombinant CD55 nucleic acid sequence.

Further provided is in view of the aforesaid, a method of improving an MSC or EV obtained from the MSC, the method comprising a step of contacting the MSC with one inflammatory factor, such as Interferon γ or TNF-α, or obtaining the EV by obtaining an EV from the MSC.

Further provided is in view of the aforesaid, an improved MSC or EV produced with a method of the invention.

The invention in view of the above-described aspects and embodiments, further pertains to the following itemized embodiments:

Item 1. A method for determining a presence or absence of an immunomodulatory activity of a mesenchymal stromal cell (MSC), wherein the method comprises a step of: detection of a presence and/or an expression of at least one biomarker selected from the group of CD55, CD271, MSCA-1 and CD146 associated with the MSC, wherein the presence and/or expression of the biomarker associated with the MSC is indicative for an immunomodulatory activity of the MSC.
Item 2. The method of item 1, wherein the detection of the presence and/or the expression comprises detecting a measurement reading from a sample derived from a potentially immunomodulatory MSC, such as the emission and/or absorption of light, the binding of a molecule, an enzymatic reaction, wherein the measurement reading is indicative for the presence of the biomarker.
Item 3. The method of item 1 or 2, wherein the method comprises the detection of the presence of two, preferably three biomarkers selected from the group CD55, CD271, MSCA-1 and CD146 associated with the MSC wherein the presence and/or expression of the two, preferably the three biomarkers is indicative for an immunomodulatory activity of the MSC.
Item 4. The method of any one of items 1 to 3, wherein the biomarker is a surface marker.
Item 5. The method of any one of items 1 to 4, wherein the immunomodulatory activity of the MSC is an immunosuppressive or immunostimulatory activity.
Item 6. The method of any one of items 1 to 5, wherein the immunomodulatory activity of the MSC is characterized by at least one of:
(i) a differentiation, activation and/or proliferation of cells associated with the innate or adaptive immune system, preferably wherein the cells are MNCs such as PBMNCs, or selected immune cell subtypes,
(ii) a presence and/or expression of an immunomodulating factor
(iii) not requiring the complement system to function.
   Item 7. The method of item 1 to 6, wherein the immunomodulating factor is selected from the group of CXCL9, IDO1, PTGS2, HLA-DRB1, CCL5, CXCL5, TNFAIP6, CD59, PTGES3L-AARSD1, IL6ST, IFNGR1, PTGDS, HPGDS, CD274, CD38, VCAM1, TICAM1, PDPN, THY1, and ADORA2A.
   Item 8. The method of items 1 to 7, wherein the immunomodulatory factor is selected from the group of HLA-DR, CD274, IDO-1, PGE2, preferably wherein the immunomodulating factor is IDO-1.
   Item 9. The method of any one of items 1 to 8, wherein the presence refers to a quantity.
   Item 10. The method of item 6 to 9, wherein the presence and/or the expression of the biomarker, and optionally of the immunomodulating factor, is compared to the presence and/or expression of a reference, preferably wherein the reference is a reference value or a reference sample.
   Item 11. The method of item 10, wherein the reference has a predetermined immunomodulatory activity, preferably wherein the predetermined immunomodulatory activity is one or more of the following:
an absence of an immunomodulatory activity,
the presence and/or expression of a biomarker,
the presence and/or expression of an immunomodulating factor,
an inhibition of PBMNC, or selected immune cell subset proliferation, and/or
the presence or absence of a measurement reading.
   Item 12. The method of any one of items 1 to 11, wherein the presence and/or expression of the biomarker is detected in the proteome and/or transcriptome of the MSC.
   Item 13. The method of any one of items 1 to 12, wherein the MSC is a bone marrow MSC, preferably a human bone marrow MSC.
   Item 14. The method of any one of items 6 to 13, wherein the MSC is cultured in a non-inflammatory environment prior to the detection of the presence of the biomarker, and optionally the detection of the presence and/or expression of the immunomodulating factor, preferably wherein the biomarker is CD55 and/or CD146, preferably CD55.
   Item 15. The method of any one of items 6 to 14, wherein the MSC is exposed to and/or cultured in an inflammatory environment prior to the detection of the presence and/or expression of the biomarker, and optionally the detection of the presence and/or expression of the immunomodulating factor, preferably wherein the biomarker is CD271 and/or MSCA-1.
   Item 16. A method for the manufacture of an immunomodulatory preparation derived from an MSC with immunomodulatory activity, wherein the method comprises determining a presence or absence of an immunomodulatory activity of an MSC according to any one of items 1 to 15.
   Item 17. The method for the manufacture of an immunomodulatory preparation derived from an MSC with immunomodulatory activity of item 16, wherein the method comprises the steps of providing a cellular preparation comprising an MSC, determining a presence or absence of an immunomodulatory activity of an MSC in the cellular preparation comprising an MSC, wherein, in the case that the presence of the immunomodulatory activity of an MSC in the cellular preparation is determined, the immunomodulatory preparation derived from an MSC with immunomodulatory activity is obtained.
   Item 18. The method of item 17, wherein the method comprises at least one additional manufacturing step after the step of determining the presence or absence of an immunomodulatory activity of the MSC in the cellular preparation comprising an MSC.
   Item 19. The method for the manufacture of an immunomodulatory preparation derived from an MSC with immunomodulatory activity of any one of items 16 to 18, wherein the method comprises a culturing of the MSC.
   Item 20. The method for the manufacture of an immunomodulatory preparation derived from an MSC with immunomodulatory activity of item 19, wherein the culturing of the MSC is conducted in an inflammatory environment.
   Item 21. The method for the manufacture of an immunomodulatory preparation derived from an MSC with immunomodulatory activity of item 20, wherein the inflammatory environment comprises a co-culture of the MSC and an allogeneic activated MNC, such an allogeneic activated PBMNC.
   Item 22. The method for the manufacture of an immunomodulatory preparation derived from an MSC with immunomodulatory activity of any one of items 16 to 21, wherein the method comprises the recombinant expression of a biomarker selected from the group of CD55, CD271, MSCA-1 and CD146.
   Item 23. The method for the manufacture of an immunomodulatory preparation derived from an MSC with immunomodulatory activity of any one of items 16 to 22, comprising a step of isolating an MSC, preferably the MSC with immunomodulatory activity, a vesicle, a micelle and/or a compound,
wherein the vesicle, micelle, or compound are associated with the MSC with immunomodulatory activity or wherein the vesicle, micelle, or compound are associated with a cell that is exposed to the MSC with immunomodulatory activity, exposed to a micelle associated with the MSC with immunomodulatory activity, exposed to a vesicle associated with the MSC with immunomodulatory activity and/or the compound associated with the MSC with immunomodulatory activity.
   Item 24. The method for the manufacture of an immunomodulatory preparation derived from an MSC with immunomodulatory activity of any one of items 16 to 23, comprising isolating a cell that is exposed to the MSC with immunomodulatory activity, exposed to a micelle associated with the MSC with immunomodulatory activity, exposed to a vesicle associated with the MSC with immunomodulatory activity and/or the compound associated with the MSC with immunomodulatory activity.
   Item 25. The method for the manufacture of an immunomodulatory preparation derived from an MSC with immunomodulatory activity of item 23 or 24, wherein the vesicle is an extracellular vesicle.
   Item 26. The method for the manufacture of an immunomodulatory preparation derived from an MSC with immunomodulatory activity of item 23 to 25, wherein the compound is a biomarker selected from CD55, CD146, MSCA-1 and/or CD271.
   Item 27. The method for the manufacture of an immunomodulatory preparation derived from an MSC with immunomodulatory activity of any one of items 23 to 26, wherein the compound is an immunomodulatory factor according to any one of items 6 to 8.
   Item 28. The method for the manufacture of an immunomodulatory preparation derived from an MSC with immunomodulatory activity of any one of items 16 to 28, wherein the step of isolating comprises isolating the MSC, preferably the MSC with immunomodulatory activity, the vesicle associated with the MSC with immunomodulatory activity, the micelle associated with the MSC with immunomodulatory activity and/or the compound associated with the MSC with immunomodulatory activity from an organism, preferably wherein the organism is a mammal.
   Item 29. An immunomodulatory preparation derived from an MSC with immunomodulatory activity, wherein the preparation comprises
the MSC with immunomodulatory activity,
a cell, vesicle, a micelle and/or a compound according to any one of items 23 to 27, and/or
a medium from a culture of the MSC with immunomodulatory activity.
   Item 30. The immunomodulatory preparation derived from an MSC with immunomodulatory activity, wherein the preparation is obtainable by the method according to any one of items 16 to 24.
   Item 31. The immunomodulatory preparation derived from an MSC with immunomodulatory activity of item 29 or 30, wherein the preparation is characterized by a presence and/or expression, preferably an increased presence and/or expression, of a biomarker selected from the group of CD55, CD146, MSCA-1 and CD271.
   Item 32. The immunomodulatory preparation derived from an MSC with immunomodulatory activity of any one of items 29 to 31, comprising one or more MSC subpopulations selected from the group of:

$$\begin{matrix}a ) & \\ MSCA 1 - CD 55 - CD 146 + CD 119 + CD 130 + PDPN - CD 140 a + CD 106 - CD 271 - & \end{matrix}$$

$$\begin{matrix}b ) & \\ MSCA 1 - CD 55 - CD 146 + CD 119 + CD 130 + PDPN - CD 140 a + CD 106 - CD 271 + & \end{matrix}$$

$$\begin{matrix}c ) & \\ MSCA 1 - CD 55 - CD 146 + CD 119 + CD 130 + PDPN - CD 140 a + CD 106 + CD 271 - & \end{matrix}$$

$$\begin{matrix}d ) & \\ MSCA 1 - CD 55 - CD 146 + CD 119 + CD 130 + PDPN + CD 140 a + CD 106 - CD 271 - & \end{matrix}$$

$$\begin{matrix}e ) & \\ MSCA 1 - CD 55 - CD 146 + CD 119 + CD 130 - PDPN - CD 140 a + CD 106 - CD 271 - & \end{matrix}$$

$$\begin{matrix}f ) & \\ MSCA 1 - CD 55 - CD 146 + CD 119 + CD 130 - PDPN - CD 140 a + CD 106 - CD 271 + & \end{matrix}$$

$$\begin{matrix}g ) & \\ MSCA 1 - CD 55 + CD 146 + CD 119 + CD 130 + PDPN - CD 140 a + CD 106 - CD 271 - & \end{matrix}$$

$$\begin{matrix}h ) & \\ MSCA 1 - CD 55 + CD 146 + CD 119 + CD 130 + PDPN + CD 140 a + CD 106 - CD 271 - & \end{matrix}$$

$$\begin{matrix}i ) & \\ MSCA 1 - CD 55 + CD 146 + CD 119 + CD 130 + PDPN + CD 140 a + CD 106 + CD 271 - & \end{matrix}$$

$$\begin{matrix}j ) & \\ MSCA 1 - CD 55 + CD 146 + CD 119 + CD 130 - PDPN + CD 140 a + CD 106 + CD 271 - & \end{matrix}$$

Item 33. The immunomodulatory preparation derived from an MSC with immunomodulatory activity of item 32, wherein the one or more MSC subpopulations is an immunomodulatory MSC subpopulation.
Item 34. An immunomodulatory preparation derived from an MSC with immunomodulatory activity according to any one of items 29 to 32 for use in the treatment of a condition or disease.
Item 35. The immunomodulatory preparation derived from an MSC with immunomodulatory activity for use of item 34, wherein the condition or disease is selected from the group of an immune disease, preferably an autoimmune disease, a graft-versus-host disease, an inflammatory disease, neurodegenerative disease and/or tissue damage.
Item 36. The immunomodulatory preparation derived from an MSC with immunomodulatory activity for use of item 34 or 35, wherein the condition or disease is selected from the group a liver disease, heart disease, bone/cartilage disease, spinal cord injury, brain disease and lung disease
Item 37. The immunomodulatory preparation derived from an MSC with immunomodulatory activity for use of any one of items 34 to 36, wherein the condition or disease is an immune disease.
Item 38. The immunomodulatory preparation derived from an MSC with immunomodulatory activity for use of any one of items 34 to 37, wherein the treatment is a regenerative treatment.
Item 39. A method for identifying an immunomodulatory population of MSCs with a known presence and/or expression of a biomarker, comprising:
(iv) co-culturing an MSC population, and allogeneic activated PBMNCs in separate or joint compartments with shared media and
(v) detecting a presence and/or expression of a biomarker of the co-cultured MSC population, preferably by transcriptome analysis and/or proteome analysis and comparing said presence and/or expression of the biomarker of the co-cultured MSC population with the presence and/or expression of the biomarker mono-cultured MSCs of the MSC population; and
(vi) detecting a presence and/or expression of an immunomodulating factor in the co-culture, preferably by transcriptome analysis and/or proteome analysis and comparing said presence and/or expression of immunomodulating factor in the co-culture with the presence and/or expression of the immunomodulating factor in a culture of mono-cultured MSCs of the MSC population; and/or
(vii) detecting the proliferation inhibition of the co-cultured allogeneic activated PBMNCs, or selected immune cell subsets, and comparing said proliferation inhibition with the proliferation of allogeneic activated PBMNCs or selected immune cell subsets, that are cultured in a separate culture that does not share media with the MSC population;
   wherein an increased presence of the immunomodulating factor, an increased expression of the immunomodulating factor and/or an increased proliferation inhibition of the allogeneic activated PBMNCs indicates an immunomodulatory MSC population; and optionally
(viii) phenotyping the MSC population regarding the presence and/or expression of said biomarker, thus resulting in the identification of immunomodulatory population of MSCs with a known presence and/or expression of a biomarker.
   Item 40. The method of item 39, wherein the MSC population originates from a sample containing MSCs only of an individual donor.
   Item 41. The method of item 39 or 40, wherein biomarker is a MSC subpopulation, or wherein the biomarker is selected from the group of CD55, CD271, MSCA-1 and CD146, preferably wherein the MSC subpopulation comprises the biomarker selected from the group of CD55, CD271, MSCA-1 and CD146.
   Item 42. The method of any one of items 39 to 41, wherein the presence and/or expression refers to a quantity.
   Item 43. The method of any one of items 39 to 42, wherein the method comprises phenotyping a MSC subpopulation in the MSC population.
   Item 44. The method of any one of items 39 to 43 wherein the method comprises an additional step of functional validation of an immunomodulatory activity of the population of MSCs.
   Item 45. The method of item 44, wherein the additional step of functional validation comprises culturing of a population of MSCs depleted or enriched of the biomarker and one or both of the following:
(i) detecting the presence and/or expression of the immunomodulating factor, in a cell lysate of the population of MSCs depleted or enriched of the biomarker and/or in the cell culture medium of the population of MSCs depleted or enriched of the biomarker;
(ii) detecting PBMNC proliferation inhibition, wherein PBMNCs, or selected immune cell subsets, are co-cultured with the population of MSCs depleted or enriched of the biomarker, preferably in the same compartment;
   wherein the presence and/or expression of the immunomodulating factor and/or the PBMNC proliferation inhibition are compared to presence and/or expression of the immunomodulating factor and/or the PBMNC proliferation inhibition of a reference, preferably wherein the reference is a population of wild-type MSCs.
   Item 46. The method of item 45, wherein the immunomodulating factor is IDO-1.
   Item 47. The method according to any one of items 39 to 46, wherein the presence and/or expression of the biomarker is correlated to the presence and/or expression of the immunomodulating factor.
   Item 48. A method for identifying an immunomodulatory subpopulation of MSCs with a known presence and/or expression of a biomarker, wherein the method is the method according to any one of items 39 to 47, wherein the immunomodulatory population of MSCs is an immunomodulatory subpopulation of MSCs and wherein the method comprises an additional step of functional validation of an immunomodulatory activity of a subpopulation of MSCs depleted or enriched of the biomarker.
   Item 49. The method of item 48, wherein the additional step of functional validation comprises culturing of the subpopulation of MSCs depleted or enriched of the biomarker and one or both of the following:
(iii) Detecting the presence and/or expression of the immunomodulating factor, preferably IDO-1, in a cell lysate of the subpopulation of MSCs depleted or enriched of the biomarker and/or in the cell culture medium of the subpopulation of MSCs depleted or enriched of the biomarker;
(iv) detecting PBMNC proliferation inhibition, wherein PBMNCs are co-cultured with the subpopulation of MSCs depleted or enriched of the biomarker, preferably in the same compartment;
   wherein the presence and/or expression of the immunomodulating factor and/or the PBMNC proliferation inhibition are compared to presence and/or expression of the immunomodulating factor and/or the PBMNC proliferation inhibition of a reference, preferably wherein the reference is a population of wild-type MSCs.
   Item 50. An agent for determining a presence or absence of an immunomodulatory activity of an MSC, wherein the agent specifically binds to a biomarker selected from the group of CD55, CD271, MSCA-1 and CD146.
   Item 51. The agent of item 50, wherein the presence of the agent and/or its binding to the biomarker is detectable using a detection method selected from spectroscopy, mass spectrometry, flow cytometry and/or a biological assay.
   Item 52. The agent of item 50 or 51, wherein the agent is a ligand molecule, an antigen binding construct or a derivative thereof specific for the biomarker, preferably wherein the antigen binding construct is a protein or nucleic acid, preferably an antibody or antigen binding fragment, a T-cell receptor, an aptamer, a nanobody, or other molecular antigen binding construct or derivative thereof specifically binding to the biomarker.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES AND SEQUENCES

The figures show:

FIG. 1: shows exemplary expressions of immunomodulating factors and biomarkers in MSCs that were co-cultured with allogeneic activated PBMNCs in separate compartments relative to their expressions in monocultured MSCs as Log 2 fold change (FC); positive values: upregulated in MSCs co-cultured with activated PBMNCs; negative values: downregulated in MSCs co-cultured with activated PBMNCs.

FIG. 2: shows flow cytometry data on the concentration of selected immunomodulating factors and biomarkers (CD59, CD90, CD271, CXCL9, HLA2, MSCA-1, CD274, CD55) samples in monocultured MSC samples and in MSC samples that were co-cultured with allogeneic activated PBMNCs in separate compartments.

FIG. 3: shows inhibition of allogeneic activated PBMNC proliferation when co-cultured with MSCs in joint compartments illustrating substantial differences in the MSCs' immunomodulation potential: 3 MSC donors with high immunosuppression potential (white columns) and 3 MSC donors with low immunosuppression potential (black columns).

FIG. 4: MSC preparations each with high and low immunosuppression potential were analyzed after monoculture, or co-culture with activated allogeneic PBMNCs by flow cytometry with a panel of 16 markers yielding 640 phenotypes. Comparative analysis of the phenotype patterns of naïve (i.e. monocultured [steady state]) and stimulated (i.e. co-cultured with activated allogeneic PBMNCs [inflammatory]) MSCs led to the identification of MSC subpopulations/stages defining MSC preparations with high or low immunosuppressive potential.

FIG. 5: shows a comparison of the inhibition of allogeneic activated PBMNC proliferation during culturing in a joint compartment with MSCs that were depleted a subpopulation with a respective biomarker (CD55, CD146 or CD271). (N=3-6 independent depletion experiments; 1-way ANOVA followed by Tukey post hoc test).

FIG. 6: shows a quantification of IDO-1 protein relative to the amount of cells in a culture of monocultured MSCs that were depleted of MSCs with the respective biomarkers (N=3 independent depletion experiments (3 donors; one depletion exp. per donor); paired t-test). (A) Amount of IDO-1 protein in the cell lysate of the culture relative to mock. (B) Amount of IDO-1 protein in conditioned medium relative to mock.

FIG. 7: shows a quantification of IDO-1 protein by flow cytometry in respective MSC subpopulations/stages identified by the distinct markers after IFN-γ, TNF-α, IL1-β stimulation. Note that the absence of CD55 and CD146 respectively dramatically reduced the percentage of IDO-1 positive MSCs from 82% (A), or 81% (B) to 55% (C) and that in the MSC subpopulation/stage positive for CD55, CD146 and MSCA-1 a very high percentage of IDO-1 positive cells (85%) could be detected (D).

The sequences show:
SEQ ID NOs. 1 to 7: amino acid sequences of CD55 isoforms
SEQ ID NOs. 8 and 9: amino acid sequences of CD271 isoforms
SEQ ID NOs. 10 and 11: amino acid sequences of CD146 isoforms
SEQ ID NOs. 12 to 14: amino acid sequences of MSCA-1 isoforms

### Examples

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

The examples show:

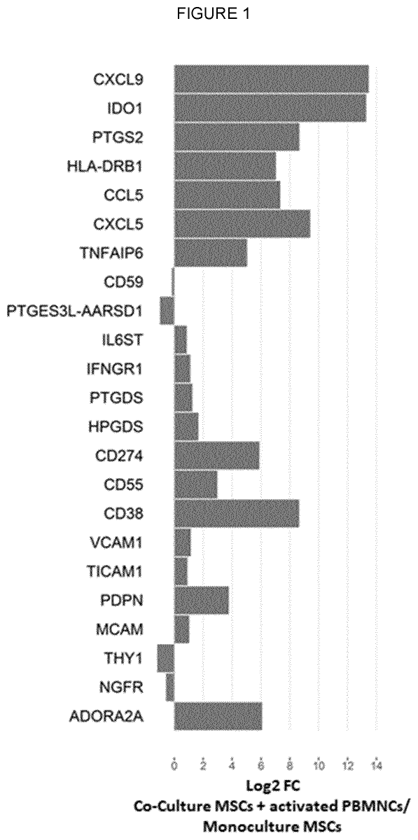

Example 1: MSCs of 5 individual donors were subjected to gene expression screening using Agilent Whole Human Genome Oligo Microarrays after monoculture (steady-state) and co-culture with allogeneic activated PBMNCs (inflammatory) in separate compartments, respectively. Analysis applying paired two-sided t-test using normalized log 2 intensity data to calculate p-values revealed a multitude of genes that were differentially (adjusted p≤0.01) expressed toward the same direction (i.e. up- or downregulated) in all donors genes under the comparative conditions (steady-state vs. inflammatory), such identifying them as IRE candidates and including novel surface proteins as candidates for suitable markers of MSC preparations with high immunomodulation potential (FIG. 1).

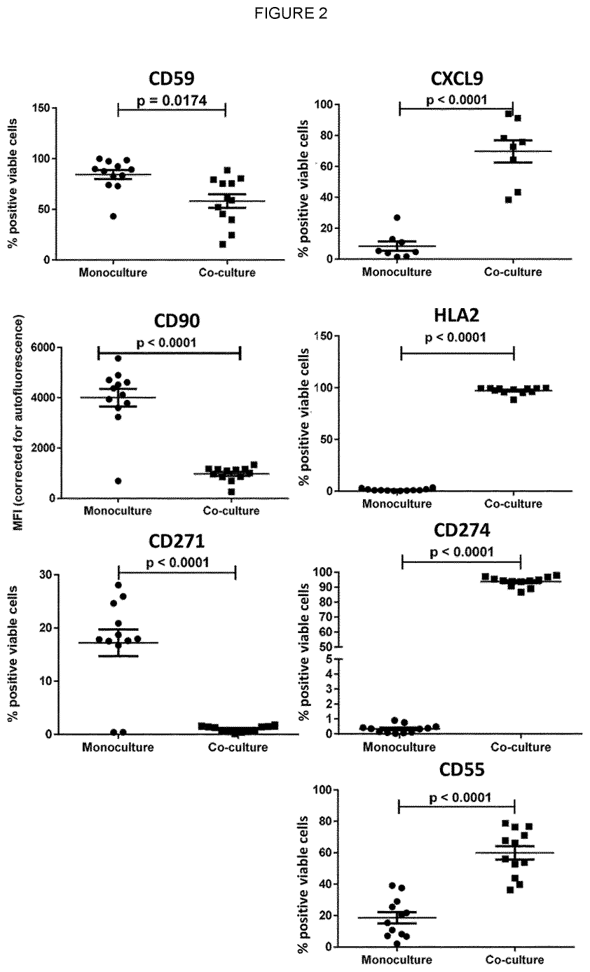

Example 2: Several under inflammatory conditions downregulated or upregulated marker candidates were validated on protein level in a second independent cohort of 5 individual MSC donors. Specifically, MSCs and pooled allogeneic PBMNCs, activated with PHA, were co-cultured in separate compartments for 4 days. In parallel, 96,000 MSCs were monocultured for 4 days, both conditions followed by flow cytometric analysis of 31 markers (FIG. 2).

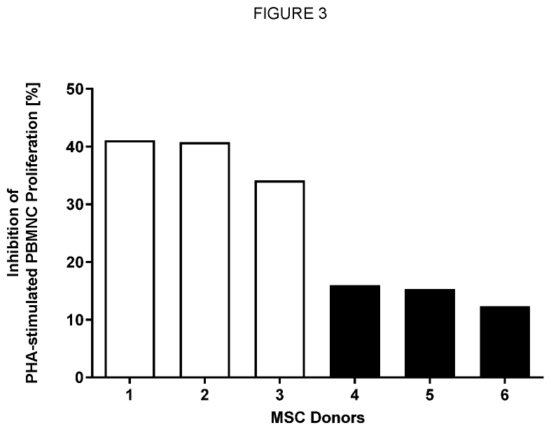

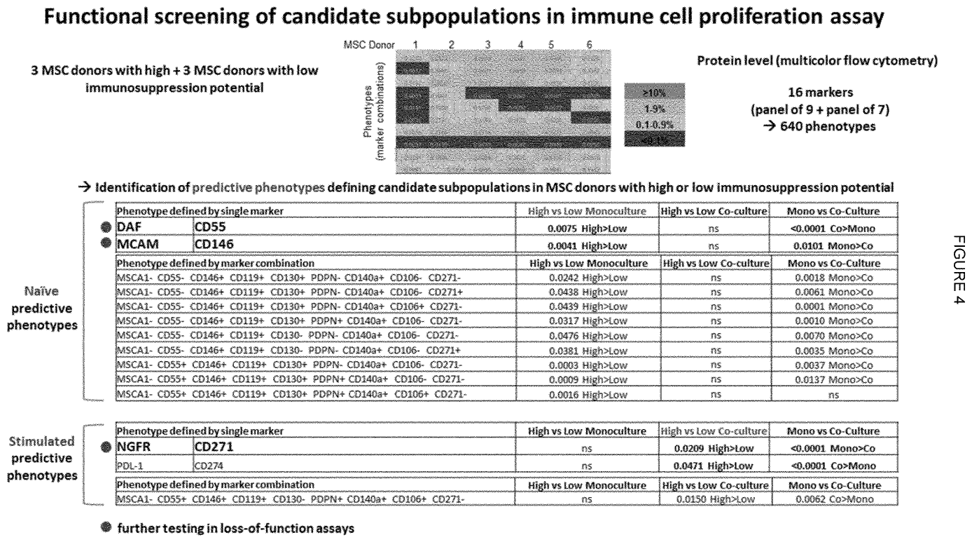

Example 3: Direct co-culture MSCs with pooled allogeneic PBMNCs, activated with PHA for 4 days; in parallel, pooled allogeneic PBMNCs, activated with PHA, were monocultured for 4 days, both conditions followed by flow cytometric analysis of PBMNC proliferation. In a first round of experiments, MSCs from 11 donors were screened with regard to their immunosuppression potential. Such, 3 MSC donors with high and 3 MSC donors with low immunosuppression potential were identified (FIG. 3). Example 4:3 MSC preparations each with high and low immunosuppression potential were subjected to separate co-culture of MSCs and pooled allogeneic PBMNCs, activated with PHA for 4 days. In parallel, MSCs were monocultured for 4 days, both conditions followed by flow cytometric analysis of a panel of 16 markers yielding 640 phenotypes. Comparative analysis of the phenotype patterns of naïve (i.e. monocultured [steady state]) and stimulated (i.e. co-cultured with activated allogeneic PBMNCs [inflammatory]) MSCs led to the identification of MSC subpopulations/stages defining MSC preparations with high or low immunosuppressive potential (FIG. 4).

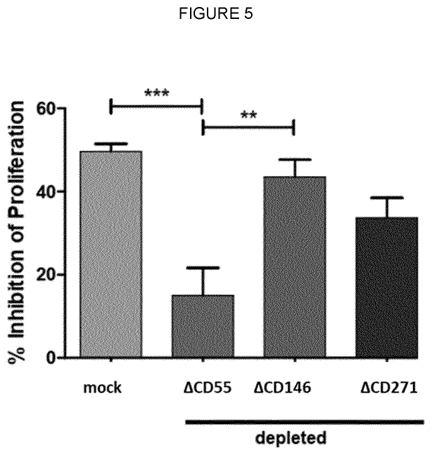

Example 5: Candidate subpopulations were functionally tested in loss-of-function experiments. MSC preparations were labelled with paramagnetic beads coated with antibodies targeting the cell surface marker of interest, followed by loading the cell suspension onto a separation column which is placed in a magnetic field. The magnetically labelled cells were retained within the column, while the unlabelled cells ran through. Thus, the resulting MSC preparation was depleted for single markers (CD55, CD146, and CD271, respectively) as confirmed by flow cytometry and analysed in direct co-culture (MSCs with pooled allogeneic PBMNCs, activated with PHA) for their potential to suppress PBMNC proliferation. For comparison MSCs were used, which were mock-depleted, i.e. processed through a separation column within a magnetic field without being magnetically labelled. The depletion of the CD55 positive MSC subpopulation/stage showed the strongest abrogation of the immunosuppressive potential of the MSC preparation such identifying the relevance of this subpopulation/stage for MSC immunomodulation (FIG. 5).

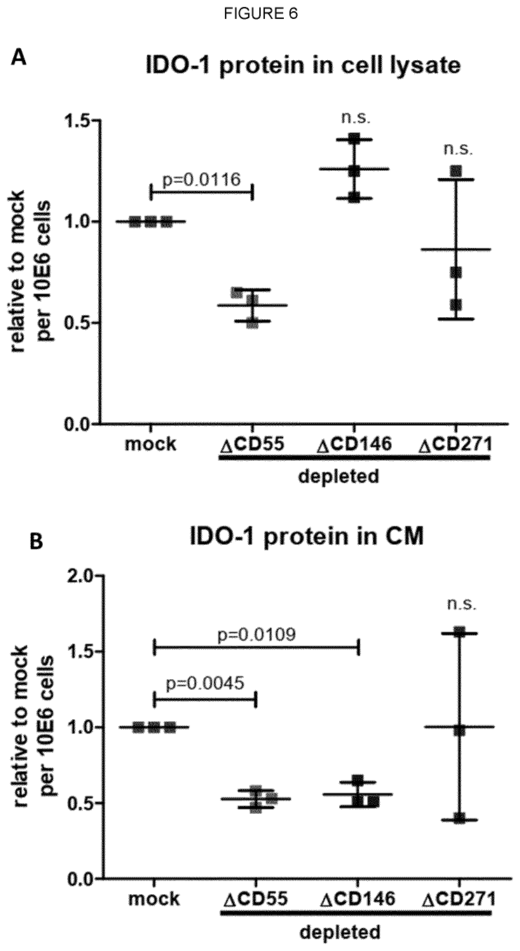

Example 6: MSC preparations were immunomagnetically depleted for CD55, or CD146, or CD271, followed by cultivation under inflammatory condition, induced by the cytokines TNF-α, IL-1β, and IFN-γ. For comparison, mock-depleted MSCs were treated accordingly. Conditioned medium (CM) was collected, cells were harvested, counted and lysed and IDO-1 protein was quantified in cell lysate and CM by ELISA. The depletion of the CD55 positive MSC subpopulation/stage showed the strongest reduction of the IDO-1 production of the MSC preparation such identifying IDO-1 as a relevant mechanism for the immunomodulation mediated by CD55 positive MSCs (FIG. 6).

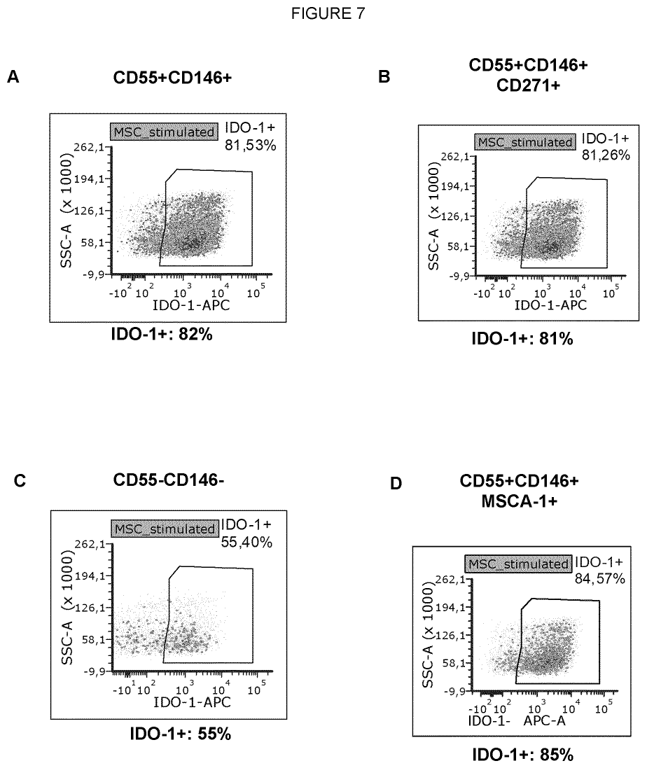

Example 7: To assess the IDO-1 protein production in distinct MSC phenotypes defined by distinct markers a flow cytometry assay was established. Hereby, MSC subpopulations/stages were identified by the distinct markers after IFN-γ, TNF-α, IL1-β stimulation. Particularly, the absence of CD55 and CD146 dramatically reduced the percentage of IDO-1 positive MSCs from 82%, or 81% to 55%. Moreover, the MSC subpopulation/stage positive for CD55, CD146 and MSCA-1 showed a very high percentage of IDO-1 positive cells (85%) (FIG. 7).

These studies identified CD55, CD146, CD271, MSCA-1 and combinations thereof as powerful predictive markers for MSC immunomodulation with IDO-1 as relevant mechanism of action.

### REFERENCES

The references are:
1. US2016120910A1
2. US2017136065A1
3. US2012027730A1
4. Soland et al, 2013, Mesenchymal Stem Cells Engineered to Inhibit Complement-Mediated Damage, PLOS ONE 8 (3): e60461, doi: 10.1371/journal.pone.0060461
5. Gavin et al, 2019, The Complement System Is Essential for the Phagocytosis of Mesenchymal Stromal Cells by Monocytes, Front. Immunol. 10:2249, doi: 10.3389/fimmu.2019.02249

1. A method for determining a presence or absence of an immunomodulatory activity of a mesenchymal stromal cell (MSC), wherein the method comprises a step of: detecting a presence and/or an expression of at least one biomarker selected from the group of CD55, CD271, MSCA-1 and CD146, wherein the presence and/or expression of the biomarker is indicative for an immunomodulatory activity of the MSC.

2. The method of claim 1, wherein the presence and/or expression of the at least one biomarker is detected in the proteome and/or transcriptome of the MSC.

3. The method of claim 1, wherein the immunomodulatory activity of the MSC is characterized by at least one of:
(i) a differentiation, activation and/or proliferation of cells associated with the innate or adaptive immune system, preferably wherein the cells are MNCs such as PBMNCs, or selected immune cell subtypes,
(ii) a presence and/or expression of an immunomodulating factor, preferably wherein the immunomodulating factor is selected from the group of CXCL9, IDO1, PTGS2, HLA-DRB1, CCL5, CXCL5, TNFAIP6, CD59, PTGES3L-AARSD1, IL6ST, IFNGR1, PTGDS, HPGDS, CD274, CD38, VCAM1, TICAM1, PDPN, THY1, and ADORA2A, HLA-DR, PGE2, most preferably wherein the immunomodulating factor is IDO-1,
(iii) not requiring the complement system to function.

4. The method of claim 1, wherein the biomarker is a surface marker.

5. The method of claim 3, wherein the presence and/or the expression of the biomarker, and optionally of the immunomodulating factor, is compared to the presence and/or expression of a reference, preferably, wherein the reference has a predetermined immunomodulatory activity, preferably wherein the predetermined immunomodulatory activity is one or more of the following:
an absence of an immunomodulatory activity,
the presence and/or expression of a biomarker,
the presence and/or expression of an immunomodulating factor,
an inhibition of PBMNC, or selected immune cell subset proliferation, and/or
the presence or absence of a measurement reading.

6. The method of claim 3, wherein the MSC is:
cultured in a non-inflammatory environment prior to the detection of the presence of the biomarker, and optionally the detection of the presence and/or expression of the immunomodulating factor, preferably wherein the biomarker is CD55 and/or CD146, preferably CD55, or
exposed to and/or cultured in an inflammatory environment prior to the detection of the presence and/or expression of the biomarker, and optionally the detection of the presence and/or expression of the immunomodulating factor, preferably wherein the biomarker is CD271 and/or MSCA-1.

7. An immunomodulatory preparation derived from an MSC with immunomodulatory activity according to claim 1, wherein the preparation comprises:
a) the MSC with immunomodulatory activity,
b) a medium from a culture of the MSC with immunomodulatory activity and/or
c) a cell, a vesicle, a micelle and/or a compound, preferably wherein the compound is a biomarker selected from CD55, CD146, MSCA-1 and/or CD271;
wherein the cell is a cell that is exposed to
i) the MSC with immunomodulatory activity,
ii) a micelle associated with the MSC with immunomodulatory activity,
iii) a vesicle associated with the MSC with immunomodulatory activity, and/or
iv) a compound associated with the MSC with immunomodulatory activity, preferably wherein the compound is a biomarker selected from CD55, CD146, MSCA-1 and/or CD271,
wherein the vesicle, micelle, and/or compound are associated with the MSC with immunomodulatory activity, or wherein the vesicle, micelle, or compound are associated with the cell that is exposed to i) to iv).

8. The immunomodulatory preparation derived from an MSC with immunomodulatory activity according to claim 7, wherein the preparation is characterized by a presence and/or expression, preferably an increased presence and/or expression, of a biomarker selected from the group of CD55, CD146, MSCA-1 and CD271.

9. The immunomodulatory preparation derived from an MSC with immunomodulatory activity of claim 7, comprising one or more MSC subpopulations selected from the group of:
$$\begin{matrix}a ) & \\ MSCA 1 - CD 55 - CD 146 + CD 119 + CD 130 + PDPN - CD 140 a + CD 106 - CD 271 - & \end{matrix}$$
$$\begin{matrix}b ) & \\ MSCA 1 - CD 55 - CD 146 + CD 119 + CD 130 + PDPN - CD 140 a + CD 106 - CD 271 + & \end{matrix}$$
$$\begin{matrix}c ) & \\ MSCA 1 - CD 55 - CD 146 + CD 119 + CD 130 + PDPN - CD 140 a + CD 106 - CD 271 - & \end{matrix}$$
$$\begin{matrix}d ) & \\ MSCA 1 - CD 55 - CD 146 + CD 119 + CD 130 + PDPN + CD 140 a + CD 106 - CD 271 - & \end{matrix}$$
$$\begin{matrix}e ) & \\ MSCA 1 - CD 55 - CD 146 + CD 119 + CD 130 - PDPN - CD 140 a + CD 106 - CD 271 - & \end{matrix}$$
$$\begin{matrix}f ) & \\ MSCA 1 - CD 55 - CD 146 + CD 119 + CD 130 - PDPN - CD 140 a + CD 106 - CD 271 + & \end{matrix}$$
$$\begin{matrix}g ) & \\ MSCA 1 - CD 55 + CD 146 + CD 119 + CD 130 + PDPN - CD 140 a + CD 106 - CD 271 - & \end{matrix}$$
$$\begin{matrix}h ) & \\ MSCA 1 - CD 55 + CD 146 + CD 119 + CD 130 + PDPN + CD 140 a + CD 106 - CD 271 - & \end{matrix}$$
$$\begin{matrix}i ) & \\ MSCA 1 - CD 55 + CD 146 + CD 119 + CD 130 + PDPN + CD 140 a + CD 106 + CD 271 - & \end{matrix}$$
$$\begin{matrix}j ) & \\ MSCA 1 - CD 55 + CD 146 + CD 119 + CD 130 - PDPN + CD 140 a + CD 106 + CD 271 - , & \end{matrix}$$
preferably, wherein the one or more MSC subpopulations is an immunomodulatory MSC subpopulation.

10. A method for the manufacture of an immunomodulatory preparation derived from an MSC with immunomodulatory activity according to claim 7, preferably wherein the method comprises determining a presence or absence of an immunomodulatory activity of an MSC.

11. The method for the manufacture of an immunomodulatory preparation derived from an MSC with immunomodulatory activity of claim 10, wherein the method comprises a culturing of the MSC, preferably wherein the culturing of the MSCs is conducted in an inflammatory environment.

12. The method for the manufacture of an immunomodulatory preparation derived from an MSC with immunomodulatory activity of claim 10, comprising a step of isolating the MSC, preferably the MSC with immunomodulatory activity, the vesicle, the micelle and/or the compound which is a biomarker selected from CD55, CD146, MSCA-1 and/or CD271.

13. A method of treatment of a condition or disease in a subject, wherein the method comprises a step of administering an immunomodulatory preparation derived from an MSC with immunomodulatory activity according to claim 7 to the subject, preferably wherein the condition or disease is selected from group of
an immune disease, preferably an autoimmune disease, a graft-versus-host disease, an inflammatory disease, neurodegenerative disease and/or tissue damage; and/or
a liver disease, heart disease, bone/cartilage disease, spinal cord injury, brain disease and lung disease.

14. The method of claim 13, wherein the treatment is a regenerative treatment, preferably wherein the regenerative treatment is a regeneration of tissue.

15. An agent for determining a presence or absence of an immunomodulatory activity of an MSC, or extracellular versicle (EV) obtained therefrom, wherein the agent specifically binds to a biomarker selected from the group of CD55, CD271, MSCA-1 and CD146.

16-19. (canceled)

20. A method for enriching an immunologically active MSC preparation or EV preparation, the method comprising providing a previously obtained MSC population or and/or EV preparation (obtained from MSC),
(i) performing a method according to claim 1 with MSCs in the previously obtained MSC or EVs;
(ii) obtaining an enriched immunologically active MSC preparation or EV preparation by combining only such MSC or EVs into a new MSC preparation or EV preparation for which a presence and/or an expression of the at least one biomarker is detected, and optionally discarding any MSC or EV for which no presence and/or an expression of the at least one biomarker is detected.

21. The method of claim 20, wherein the at least one biomarker is CD55.

22. The method of claim 20, wherein the presence and/or an expression of the at least one biomarker is detected compared to a reference value, preferably wherein the reference value is a negative or positive control reference.

23. A method of producing a genetically modified MSC, the method comprising a step of introducing into the MSC an expressible sequence encoding a recombinant CD55 protein, optionally expressing the recombinant CD55 protein in the genetically modified MSC.

24. A genetically modified MSC or an EV obtained therefrom, wherein the genetically modified MSC or EV obtained therefrom comprises a recombinantly expressed CD55 protein, preferably wherein the recombinantly expressed CD55 is expressed by a genetically introduced recombinant CD55 nucleic acid sequence.

25. A method of improving an MSC or EV obtained from the MSC, the method comprising a step of contacting the MSC with one inflammatory factor, such as Interferon γ or TNF-α, or obtaining the EV by obtaining an EV from the MSC.

26. (canceled)


SEQUENCE LISTING (14 sequences)

SEQ ID NO: 1 - protein, 381 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 2 - protein, 440 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGSRPVTQAGMRWCDRSSLQSRTPGFKRSFHFSLPSSWYYRAHVFHVDRFAWDASNHGLADLAKEELRRKYTQVYRLFLVS

SEQ ID NO: 3 - protein, 384 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSALQVRPFEVSGSSHISSKKMMCIL

SEQ ID NO: 4 - protein, 364 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSVLFM

SEQ ID NO: 5 - protein, 439 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSETVFHRVIQDGLDLLASRSACLGLPKCWDYRREPPHLARAHVFHVDRFAWDASNHGLADLAKEELRRKYTQVYRLFLVS

SEQ ID NO: 6 - protein, 525 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQGTETPSVLQKHTTENVSATRTPPTPQKPTTVNVPATIVTPTPQKPTTINVPATGVSSTPQRHTIVNVSATGTLPTLQKPTRANDSATKSPAAAQTSFISKTLSTKTPSAAQNPMMTNASATQATLTAQKFTTAKVAFTQSPSAAPTRSTPVSRTTKHFHETTPNKGSGTTSGTTRLLSGHTCFTLTGLLGTLVTMGLLT

SEQ ID NO: 7 - protein, 551 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQGTETPSVLQKHTTENVSATRTPPTPQKPTTVNVPATIVTPTPQKPTTINVPATGVSSTPQRHTIVNVSATGTLPTLQKPTRANDSATKSPAAAQTSFISKTLSTKTPSAAQNPMMTNASATQATLTAQKFTTAKVAFTQSPSAARKSTNVHSPVTNGLKSTQRFPSAHITATRSTPVSRTTKHFHETTPNKGSGTTSGTTRLLSGHTCFTLTGLLGTLVTMGLLT

SEQ ID NO: 8 - protein, 427 aa, Homo sapiens
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 9 - protein, 333 aa, Homo sapiens
MSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 10 - protein, 646 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQGLDLDTMISLLSEPQELLVNYVSDVRVSPAAPERQEGSSLTLTCEAESSQDLEFQWLREETGQVLERGPVLQLHDLKREAGGGYRCVASVPSIPGLNRTQLVNVAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRGVVIVAVIVCILVLAVLGAVLYFLYKKGKLPCRRSGKQEITLPPSRKSELVVEVKSDKLPEEMGLLQGSSGDKRAPGDQGEKYIDLRH

SEQ ID NO: 11 - protein, 527 aa, Homo sapiens
MVYIVRQFLLYNVSGSVYLDQLIVLLTAKFSILRIAGSRVHHSPFSGHLDGCSFLSLQHSLHTSLDMSRHENVFLGLTLSSKSAGLKGFQLAFVPGLLQGTGGYLDGPLPTPVDNPRVGLEVGLRLSLPPLPPCPGVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQGLDLDTMISLLSEPQELLVNYVSDVRVSPAAPERQEGSSLTLTCEAESSQDLEFQWLREETGQVLERGPVLQLHDLKREAGGGYRCVASVPSIPGLNRTQLVNVAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTGKPGLAREQGCARASFLPCPSPESPVQKGE

SEQ ID NO: 12 - protein, 524 aa, Homo sapiens
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

SEQ ID NO: 13 - protein, 447 aa, Homo sapiens
MPWSFRSSTPTWLRMSSCSWEMTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

SEQ ID NO: 14 - protein, 469 aa, Homo sapiens
MFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF